SEQUENCE LISTING (11 sequences)

SEQ ID NO: 1 - protein, 133 aa, Artificial Sequence
  source: /note=“Description of Artificial Sequence Synthetic polypeptide”
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 205 aa, Artificial Sequence
  source: /note=“Description of Artificial Sequence Synthetic polypeptide”
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 3 - protein, 155 aa, Streptococcus agalactiae
MKFEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDCTQDIFIANKTDIEKNIWMLQAKLGKAPGIDA

SEQ ID NO: 4 - protein, 153 aa, Streptococcus agalactiae
MVYEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGL

SEQ ID NO: 5 - protein, 165 aa, Artificial Sequence
  source: /note=“Description of Artificial Sequence Synthetic polypeptide”
MKFEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDSTQDIFIANKTDIEKNIWMLQAKLGKAPGIDAGSSGHHHHHH

SEQ ID NO: 6 - protein, 163 aa, Artificial Sequence
  source: /note=“Description of Artificial Sequence Synthetic polypeptide”
MVYEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGLGSSGHHHHHH

SEQ ID NO: 7 - protein, 185 aa, Artificial Sequence
  source: /note=“Description of Artificial Sequence Synthetic polypeptide”
MTNTLVENIYASVTHNISKKEASKNEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGLGSSGHHHHHH

SEQ ID NO: 8 - protein, 25 aa, Streptococcus pyogenes
MTNTLVENIYASVTHNISKKEASKN

SEQ ID NO: 9 - protein, 10 aa, Artificial Sequence
  source: /note=“Description of Artificial Sequence Synthetic peptide”
GSGSGSGSGS

SEQ ID NO: 10 - protein, 5 aa, Artificial Sequence
  source: /note=“Description of Artificial Sequence Synthetic peptide”
GSSGH

SEQ ID NO: 11 - protein, 221 aa, Artificial Sequence
  source: /note=“Description of Artificial Sequence Synthetic polypeptide”
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGSGSGSHHHHHH

(19) United States
(12) Patent Application Publication
(10) Pub. No.: US 2023/0210975 A1
(43) Pub. Date: Jul. 6, 2023

(54) BACTERIAL IMMUNIZATION USING NANOPARTICLE VACCINE

(71) Applicant: GLAXOSMITHKLINE BIOLOGICALS SA, Rixensart (BE)

(72) Inventors: Roberto ADAMO, Siena (IT); Filippo CARBONI, Siena (IT); Roberta COZZI, Siena (IT); Immaculada MARGARIT Y ROS, Siena (IT); Maria Rosaria ROMANO, Siena (IT)

(73) Assignee: GLAXOSMITHKLINE BIOLOGICALS SA, Rixensart (BE)

(21) Appl. No.: 18/008,777

(22) PCT Filed: Jun. 11, 2021
(86) PCT No.: PCT/IB2021/055159

§ 371 Date: Dec. 7, 2022

Related U.S. Application Data
(60) Provisional application No. 63/038,277, filed on Jun. 12, 2020.

(51) Int. Cl. A61K 39/09; A61P 31/04

(52) U.S. Cl. CPC A61K 39/092; A61P 31/04; A61K 2039/6031

(57) ABSTRACT

Methods of inducing an immunogenic response against a bacterial polysaccharide or oligosaccharide, and constructs and compositions for use in such methods.

### SEQUENCE LISTING

The instant application contains a Sequence Listing which has been submitted electronically in ASCII format and is hereby incorporated by reference in its entirety. Said ASCII copy, created on Jun. 7, 2021, is named VU66934 WO_SL.txt and is 13,971 bytes in size.

### FIELD OF THE INVENTION

This invention relates to methods of inducing an immunogenic response against a bacterial polysaccharide or oligosaccharide, and constructs and compositions for use in such methods.

### BACKGROUND

Streptococcus agalactiae (also known as “Group B Streptococcus” or “GBS”) is a β-hemolytic, encapsulated Gram-positive microorganism that is a major cause of neonatal sepsis and meningitis, particularly in infants born to women carrying the bacteria (Heath & Schuchat (2007)). The use of intrapartum antibiotic prophylaxis has reduced early-onset neonatal disease but has not significantly affected the incidence of late-onset (7-90 days after birth) neonatal GBS disease (see, e.g., Baker (2013)). An effective vaccine designed for maternal administration during pregnancy is desirable to prevent GBS disease in infants; currently no licensed GBS vaccine is available.

The GBS capsule is a virulence factor that assists the bacterium in evading human innate immune defences. The GBS capsule consists of high molecular weight polymers made of multiple identical repeating units of four to seven monosaccharides and including sialic acid (N-acetylneuraminic acid) residues, referred to as Capsular Polysaccharides (CPS). GBS can be classified into ten serotypes (Ia, Ib, II, III, IV, V, VI, VII, VIII, and IX) based on the chemical composition and the pattern of glycosidic linkages of the capsular polysaccharide repeating units. Non-typeable strains of GBS are also known to exist. Description of the structure of GBS CPS may be found in the published literature (see e.g., WO2012/035519). The capsular polysaccharides of different GBS serotypes are chemically related but are antigenically different.

GBS capsular polysaccharides (also referred to as capsular saccharides) have been investigated for use in vaccines. However, saccharides are T-independent antigens and are generally poorly immunogenic. Covalent conjugation of a saccharide to a carrier molecule (such as a monomeric protein carrier) can convert T-independent antigens into T-dependent antigens, thereby enhancing memory responses and allowing protective immunity to develop Immune interference is a concern where a subject receives multiple different vaccines (either concurrently or sequentially) that contain the same carrier protein (see, e.g., Findlow and Borrow (2016); Voysey et al., (2016); Dagan et al., Infect. Immun 66:2093-2098 (1998)). Tetanus toxoid (TT), diphtheria toxoid (DT), and cross-reacting material 197 (CRM, or CRM197) are currently used as monomeric carrier proteins in marketed vaccines against H. influenzae and multiple strains of meningococcal bacteria. CRM197 is additionally found in marketed multivalent pneumococcal vaccines.

GBS glycoconjugates of CPS serotypes Ia, Ib, II, III, IV and V conjugated to monomeric carrier proteins have separately been shown to be immunogenic in humans. Multivalent GBS vaccines have been described, e.g., in WO2016-178123, WO2012-035519, and WO2014-053612. Clinical studies using monovalent or bivalent GBS glycoconjugate (saccharide+carrier protein) vaccines have previously been conducted with both non-pregnant adults and pregnant women. See, e.g., Paoletti et al. (1996); Baker et al. (1999); Baker et al., (2000); Baker et al. (2003); Baker et al. (2004).

A pentavalent GBS glycoconjugate vaccine (serotypes Ia, Ib, II, III, and V, conjugated to monomeric carrier protein, and with or without aluminum phosphate adjuvant) has been evaluated in a phase I trial (NCT03170609). A GBS trivalent vaccine (serotypes Ia, Ib, and III) comprising conjugates of GBS CPS and the monomeric carrier protein CRM197 was evaluated for use in maternal vaccination in a phase 1b/2 clinical trial (NCT01193920); infants born to the vaccinated women were reported to have higher GBS serotype-specific antibody levels (transplacentally transferred antibodies) until 90 days of age, compared with a placebo group (Madhi et al., Clin. Infect. Dis. 65(11):1897-1904 (2017).

Typical monomeric carrier proteins used for the conjugation with bacterial saccharide antigens for the development of potential vaccines are the Tetanus Toxoid (TT), the genetically detoxified diphtheriae toxoid (CRM197) and GBS pili proteins (Nilo et al, (2015a) and Nilo et al, (2015b)).

There is a continuing need for antigenic constructs, and compositions comprising such constructs, that are capable of inducing an immune response against GBS and other bacterial pathogens in human subjects.

### SUMMARY OF THE INVENTION

A first embodiment of the present invention is a protein nanoparticle, such as a non-viral protein nanoparticle or a Virus-Like Particle (VLP), having antigenic molecules conjugated to its exterior surface, where the antigenic molecules are bacterial saccharides, such as polysaccharides or oligosaccharides. The bacterial saccharides may be capsular saccharides or O-antigen saccharides. The bacterial saccharide, such as polysaccharide or oligosaccharide, may be selected from a bacterial species selected from the group consisting of a Acinetobacter species, Bacillus species, Bordetella species, Borrelia species, Burkholderia species, Campylobacter species, Candida species, Chlamydia species, Clostridium species, Corynebacterium species, Enterococcus species, Escherichia species, Francisella species, Haemophilus species, Helicobacter species, Klebsiella species, Legionella species, Listeria species, Neisseria species, Proteus species, Pseudomonas species, Salmonella species, Shigella species, Staphylococcus species, Streptococcus species, Streptomyces species, Vibrio species, and Yersinia species.

In one embodiment of the invention, the protein nanoparticle is a Virus Like Particle (VLP) made of viral protein subunits.

In one embodiment of the invention, the VLP is a QBeta VLP.

In one embodiment of the invention, the protein nanoparticle is a non-viral nanoparticle made of non-viral protein subunits.

In one embodiment, the non-viral nanoparticle is a ferritin nanoparticle or a mI3 nanoparticle.

In a further embodiment of the invention, the VLP is a QBeta VLP having bacterial capsular polysaccharides or oligosaccharides conjugated to the exterior surface of the VLP.

A further embodiment of the invention is an immunogenic composition or pharmaceutical composition comprising a nanoparticle, such as a non-viral nanoparticle or a VLP of the invention.

In a further embodiment, the nanoparticle of the invention, such as non-viral nanoparticle or VLP, immunogenic composition, or pharmaceutical composition of the invention is used for the manufacture of a medicament for inducing an immune response, or used to induce an immune response in a subject.

A further embodiment of the invention is a method of inducing an immune response in a human subject, by administering to the subject an immunologically effective amount of the nanoparticle, such as non-viral nanoparticle or VLP, immunogenic composition, or pharmaceutical composition of the invention.

### BRIEF DESCRIPTION OF THE FIGURES

FIG. 1 illustrates depolymerization of GBS serotype II capsular polysaccharides to provide shorter oligosaccharides by: de-N-Acetylation with NaOH, oxidation with NaNO₂, and N-acetylation, followed by purification using a desalting column.

FIG. 2 illustrates modification of GBS serotype II short oligosaccharides with a hydrazine linker (ADH) and an active ester spacer (SIDEA).

FIG. 3 depicts the conjugation of modified GBS serotype II short oligosaccharides (as shown in FIG. 2) to NPs.

FIG. 4 illustrates oxidation of GBS serotype II capsular polysaccharide using NaIO₄.

FIG. 5 depicts the conjugation of modified GBS serotype II polysaccharides (as shown in FIG. 3) to NPs.

FIG. 6 provides a graph of SE-HPLC analysis of GBS OSII-ferritin NP conjugate, and GBS ferritin NP (without conjugated saccharide).

FIG. 7 provides a graph of SE-HPLC analysis of GBS PSII-ferritin NP conjugate, and GBS ferritin NP (without conjugated saccharide).

FIG. 8 provides a graph of SE-HPLC analysis of GBS OSII-mI3 NP conjugate, and mI3 NP (without conjugated saccharide).

FIG. 9 provides a graph of SE-HPLC analysis of GBS PSII-mI3 NP conjugate, and mI3 NP (without conjugated saccharide).

FIG. 10 provides a graph of SE-HPLC analysis of GBS OSII-QBeta NP conjugate, and QBeta NP (without conjugated saccharide).

FIG. 11 provides a graph of SE-HPLC analysis of GBS PSII-QBeta NP conjugate, and QBeta NP (without conjugated saccharide).

FIG. 12A shows results of SDS-PAGE (4-12% in MOPS), where lane 1 is GBS ferritin NP, lane 2 is OSII-GBS ferritin NP, lane 3 is PSII-GBS ferritin NP, lane 4 is mI3 NP, lane 5 is OSII-mI3 NP, lane 6 is PSII-mI3 NP, lane 7 is QBeta nanoparticle, lane 8 is OSII-QBeta NP, and land 9 is PSII-QBeta NP.

FIG. 12B provides Western Blot results, where lane 1 is GBS ferritin NP, lane 2 is OSII-GBS ferritin NP, lane 3 is PSII-GBS ferritin NP, lane 4 is mI3 NP, lane 5 is OSII-mI3 NP, lane 6 is PSII-mI3 NP, lane 7 is QBeta nanoparticle, lane 8 is OSII-QBeta NP, and land 9 is PSII-QBeta NP.

FIG. 13 provides a graph of SE-HPLC analysis of GBS PSIa-QBeta NP conjugate, and QBeta NP (without conjugated saccharide).

FIG. 14 provides negative stain TEM image of QBeta nanoparticles conjugated to GBS PSIa, showing typical icosahedral symmetry with a diameter around 33 nm. (Scale bat=200 nm)

FIG. 15 illustrates the process of preparing conjugates of Streptococcus pneumonia polysaccharide serotype 12F with QBeta nanoparticles or monomeric protein carrier CRM197.

FIG. 16 provides the SE-HPLC analysis of S. pneumoniae PS12F-QBeta NP conjugate and the QBeta NP (no conjugated saccharide).

FIG. 17 provides negative stain TEM image of QBeta nanoparticles conjugated to S. pneumoniae PS12F.

### SEOUENCES

| SEQ ID | | |
|---|---|---|
| NO | Description | Length |
| 1 | QBeta VLP subunit sequence | 133 aa |
| 2 | Mi3 sequence | 205 aa |
| 3 | GBS ferritin from DK-PW-092 strain (“GBS 092”) | 155 aa |
| 4 | GBS ferritin Strain 14747 (“GBS 14747”) | 153 aa |
| 5 | GBS 092 (Cys to Ser at #124) + C-terminal His Tag | 165 aa |
| 6 | GBS 14747 + C-terminal His Tag | 163 aa |
| 7 | GBS 14747 + N-term helix + C-term His Tag | 185 aa |
| 8 | N-terminal helix from S. pyogenes (Group A strep) | 25 aa |
| 9 | Peptide linker GSGSGSGSGS | 10 aa |
| 10 | Peptide linker GSSGH | 5 aa |
| 11 | MI3 sequence + linker + His tag | 221 aa |

### DETAILED DESCRIPTION

The present invention relates to self-assembling protein nanoparticles (also referred to herein as nanoparticles, or NPs that display bacterial capsular polysaccharide or oligosaccharide antigenic molecules on the external nanoparticle surface, to compositions comprising such nanoparticles, and to methods of using such nanoparticles and compositions.

The NPs used in the present invention are capable of self-assembly from subunit proteins, into nanoparticles, i.e., particles of less than about 100 nm in maximum diameter. Self-assembly of NPs refers to the oligomerization of polypeptide subunits into an ordered arrangement, driven by non-covalent interactions. In one embodiment of the invention, multiple copies of structurally defined antigenic epitopes are displayed on the exterior surface of the NP.

NPs may be derived from non-viral protein subunits (non-viral NPs) or protein subunits derived from viral or bacteriophage protein subunits (viral-like particles, or VLPs).

In one embodiment, the NP of the present invention is a QBeta (Qβ) bacteriophage VLP. In a specific embodiment the QBeta VLP is conjugated to a capsular polysaccharide or oligosaccharide antigen on the external NP surface. In another embodiment, the NP of the present invention is a GBS ferritin or mI3 nanoparticle displaying bacterial capsular polysaccharide or oligosaccharide antigens on the external NP surface.

The bacterial capsular polysaccharide or oligosaccharide may be selected from the group consisting of a Acinetobacter species, Bacillus species, Bordetella species, Borrelia species, Burkholderia species, Campylobacter species, Candida species, Chlamydia species, Clostridium species, Corynebacterium species, Enterococcus species, Escherichia species, Francisella species, Haemophilus species, Helicobacter species, Klebsiella species, Legionella species, Listeria species, Neisseria species, Proteus species, Pseudomonas species, Salmonella species, Shigella species, Staphylococcus species, Streptococcus species, Streptomyces species, Vibrio species, and Yersinia species.

The NPs of the present invention may be used for any suitable purpose, such as for inducing an immune response in a subject.

The present inventors have surprisingly found that NPs displaying bacterial capsular polysaccharide or oligosaccharide antigens efficiently induced specific immune responses, in particular antibody responses. Such responses could be induced in the absence of an adjuvant. Using certain NP constructs, strong immune responses to displayed bacterial capsular polysaccharide or oligosaccharide antigens were achieved after a single administration, and were higher than the responses induced by a single administration of a the bacterial saccharide-CRM197 conjugate. Some NP constructs displaying bacterial saccharide antigens induced in mice after one dose a comparable or higher immune response compared to two doses of the bacterial saccharide-CRM197 conjugates.

Accordingly, the present invention provides a NP conjugated to a GBS saccharide antigen, such as a polysaccharide or oligosaccharide antigen, wherein the NP is capable of inducing an immune response to the saccharide antigen following a single dose, and wherein the immune response is higher than the immune response elicited by a single dose of a monomeric protein carrier, such as CRM197, displaying the same GBS saccharide.

In another embodiment, the NP is capable of inducing an immune response to the GBS saccharide antigen following a single dose, wherein the immune response is comparable to the immune response elicited by two doses of a monomeric protein carrier, such as CRM197, displaying the same GBS saccharide.

The present invention provides glycoconjugate vaccines against a variety of bacterial pathogens that present cell surface carbohydrates, including GBS, S. pneumoniae, K. pneumoniae, E. coli, S. aureus and others, where an effective immune response may be achieved after a single administration.

### NPs and VLPs

Protein NPs, including non-viral NPs and VLPs, have been described as scaffolds to present antigens linked thereto in highly ordered repetitive antigen arrays (see e.g., WO02/056905). VLPs are supermolecular structures built from multiple viral protein molecules (polypeptide subunits) of one or more types. VLPs lack the viral genome and are therefore noninfectious. VLPs can often be produced in large quantities by recombinant expression methods.

Examples of VLPs include those made of the viral capsid proteins of hepatitis B virus (Ulrich, et al., (1998)), measles virus (Wames, et al., Gene 160:173-178 (1995)), Sindbis virus, rotavirus (U.S. Pat. Nos. 5,071,651 and 5,374,426), foot-and-mouth-disease virus (Twomey, et al., (1995)), Norwalk virus (Jiang, et al., (1990); Matsui, et al., J. Clin. Invest. 87:1456-1461 (1991)), the retroviral GAG protein (WO 96/30523), the surface protein of Hepatitis B virus (WO 92/11291), and human papilloma virus (WO 98/15631).

VLPs may also be made from recombinant proteins of an RNA-phage, such as from bacteriophage QBeta, bacteriophage R17, bacteriophage fr, bacteriophage GA, bacteriophage SP, bacteriophage MS2, bacteriophage M11, bacteriophage MX1, bacteriophage NL95, bacteriophage f2, and bacteriophage PP7.

Nanoparticles made of non-viral protein subunits have also been reported as able to display antigenic molecules on the exterior surface. Such NPs include those made of bacterial, insect, and mammalian proteins that naturally self-assemble into NPs. Bacterial lumazine synthase (LS) has been investigated for use as an antigen-carrying protein particle. Jardine et al. (2013) reported LS from the bacterium Aquifex aeolicus fused to an HIV gp120 antigen self-assembled into a 60-mer nanoparticle. Nucleotide sequences encoding fusions of bacterial (H. pylori) ferritin subunit polypeptide and protein antigens have been described, e.g., for rotavirus antigens, influenza antigens, and N. gonorrhoeae antigens (Li et al., (2019); Kanekiyo et al., (2013); Wang et al., (2017)). Recombinant expression and self-assembly into NPs displaying the antigenic peptides on the NP exterior surface are reported. Nanoparticles based on insect and human ferritin have also been described for use in displaying, on the NP surface, antigens (see, e.g., WO2018/005558; Kwong et al. (2018); Li et al., (2006)).

Ferritin and ferritin-like proteins from GBS strains have been shown to self-assemble into a 12-mer nanoparticle when recombinantly expressed. These GBS proteins show homology to the S. pyogenes DPS-like peroxide resistance polypeptide subunit (see, e.g., GenBank KLL27267.1, protein from GBS DK-PW-092 strain (SEQ ID NO: 3); protein from GBS Strain 14747 (SEQ ID NO: 4)). The GBS ferritin or ferritin-like subunit sequence may be recombinantly modified to contain a short amino acid tag to aid in purification, such as a histidine tag as is known in the art, which may be joined to the ferritin or ferritin-like sequence via a short peptide linker (see SEQ ID NO: 6, which is SEQ ID NO: 4 with a C-terminal histidine tag joined by a peptide linker). GBS ferritin or ferritin-like proteins may also be modified to replace naturally-occurring cysteine residues, where modeling suggests the cysteine will not establish an intramolecular or intra-nanoparticle disulfide bridge. Replacement of such cysteine residues, e.g., with a serine residue, may avoid potential aggregation during production of NPs. Replacing the cysteine residue at amino acid position 124 of SEQ ID NO: 3 provides SEQ ID NO: 5, which also contains a C-terminal histidine tag. Additionally, the GBS ferritin or ferritin-like molecules may be modified to contain an N-terminal helical portion to improve the colloidal stability and yield of NPs. For example, SEQ ID NO: 7 is a modification of SEQ ID NO: 4 (strain 14747), where the first (N-terminal) three amino acids of SEQ ID NO: 4 are replaced with the N-terminal 25 amino acids of S. pyogenes Dpr (SEQ ID NO: 8), to provide an N-terminal Helix. SEQ ID NO: 7 also comprises a C-terminal Histidine tag. Such modified GBS ferritin or ferritin-like polypeptides maintain the ability to self-assemble into a nanoparticle protein, such as a nanoparticle made up of twelve copies of the same modified subunit polypeptide. Recombinant production of any of SEQ ID NO: 5, SEQ ID NO: 6, or SEQ ID NO: 7, can be used to provide NPs. Lysine or asparagine residues exposed at the surface of the NP may be used in conjugating glycans to the NP surface.

In specific embodiments are provided a protein nanoparticle comprising a subunit polypeptide having at least 95%, at least 96%, at least 97%, at least 98% at least 99% or 100% sequence identity to any one of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 11, wherein the subunit protein is capable of self-assembling to form the nanoparticle.

Protein NPs, including non-viral NPs andVLPs, may be produced by recombinant gene expression in a prokaryotic expression system. Viral capsid proteins have been shown to efficiently self-assemble to form VLPs upon expression in a bacterial host. U.S. Pat. No. 9,657,065 describes a process for the purification recombinantly expressed, self-assembled VLP from the homogenate of a bacterial host, wherein the VLPs were produced by expression of viral capsid proteins in the bacterial host.

In one preferred embodiment, the present invention utilizes a NP which is a VLP made of coat proteins from the E. coli RNA bacteriophage QBeta (QBeta). QBeta VLPs have an essentially icosahedral phage-like capsid structure with a diameter of about 25 nm. The capsid contains 180 copies of coat protein, linked in covalent pentamers and hexamers by disulfide bridges (Golmohammadi, et al., (1996)). Capsids or VLPs made from recombinant QBeta coat proteins may contain, however, subunits which are either not linked via disulfide bonds to other subunits within the capsid, or which are incompletely linked, meaning that such VLPs comprise fewer than the maximum number of possible disulfide bonds.

The gene for the QBeta coat protein (CP) contains a “leaky” stop codon that occasionally results in a readthrough by the host ribosome producing a minor coat protein A₁. A₁ consists of the full-length coat domain connected by a flexible linker to the readthrough domain, a 196-amino acid C-terminal extension (Cui et al., (2017); Runnieks et al. (2011)).

QBeta capsid proteins used to produce VLPs may include QBeta Coat Protein (CP) and QBeta A1 protein, and variants thereof, including variant proteins in which the N-terminal methionine is cleaved; C-terminal truncated forms of QBeta A1 missing as much as 100, 150 or 180 amino acids; variant proteins which have been modified by the removal of a lysine residue by deletion or substitution or by the addition of a lysine residue by substitution or insertion (see for example QBeta-240, QBeta-243, QBeta-250, QBeta-251 and QBeta-259 disclosed in WO03/024481 (U.S. Pat. No. 8,691,209; U.S. Pat. No. 9,950,055)). See also, e.g., WO02/056905, WO03/024480. Typically the percentage of QBeta A1 protein relative to CP in the VLP is limited, to ensure VLP formation. See QBeta Coat Protein (CP) Protein Information Resource (PIR) Database, Accession No. VCBPQBeta; QBeta A1 protein PIR Database Accession No. AAA16663.

VLPs of QBeta, and methods for their preparation, are provided in WO 02/056905. QBeta CP can self-assemble into capsids when expressed recombinantly in E. coli (Kozlovska et al., (1993)), though the N-terminal methionine of QBeta CP may be removed (Stoll et al., (1977)). VLPs composed of QBeta CPs where the N-terminal methionine has not been removed, or VLPs comprising a mixture of QBeta CPs where the N-terminal methionine is either cleaved or present, are useful within the scope of the present invention.

Recombinant QBeta VLPs produced using recombinant gene expression in a bacterial expression system may be purified from bacterial homogenate by size exclusion chromatography (Kozlovska et al. 1993) or by a combination of fractionated ammonium sulphate precipitation and size exclusion chromatography (Vasiljeva et al (1998); Ciliens et al. (2000)).

VLPs of QBeta coat proteins display lysine residues on their surface. VLPs of QBeta mutants, where exposed lysine residues are replaced by arginines are also useful in the present invention.

One embodiment of the present invention uses NPs or VLPs consisting of or comprising QBeta CP, where the CP comprises SEQ ID NO:1 (133 amino acids including methionine in position 1) or consisting of or comprising amino acids 2-133 of SEQ ID NO:1 (excludes the initial methionine).

Hsia et al. (2016) describe the computational design of an icosahedral nanoparticle that self-assembles from trimeric building blocks (i301). The i301 nanocage is based on the 2-keto-3-deoxy-phosphogluconate (KDPG) aldolase from the hyperthermophilic bacterium Thermotoga maritima; i301 has five mutations compared to the wild-type protein, and assembles into a higher order dodecahedral 60-mer. The i301 sequence was further altered (two cysteine to alanine substitutions, C76A and C100A)) to provide the “mi3” sequence (SEQ ID NO:2), which is also capable of assembling into 60-mer nanoparticles. Bruun et al. (2018).

### Polypeptides and NPs

VLP or NP subunit polypeptides may contain an amino acid sequence known as a “tag”, which facilitates purification (e.g. a polyhistidine-tag to allow purification on a nickel-chelating resin). Examples of affinity-purification tags include, e.g., 6×His tag (hexahistidine, binds to metal ion), 8×His tag; maltose-binding protein (MBP) (binds to amylose), glutathione-S-transferase (GST) (binds to glutathione), or other tags as are known in the art. In certain embodiments, the tag may be linked directly at the N-terminus of VLP or NP subunit polypeptide, or attached thereto by a short polypeptide linker sequence. Suitable polypeptide linkers include linkers of two or more amino acids. An illustrative polypeptide linker is one or more multimers of GGS or GSS, or variations thereof such as GGSGG (SEQ ID NO: 39) or GSGGG (SEQ ID NO: 63). Several (such as 1, 2, 3, 4, 5, 6, 7, 8, 9 or 10) N-terminal amino acid residues of the NP subunit polypeptide sequence may be deleted and replaced with the linker sequence. The tag may be removed (enzymatically or through other means) prior to NP or VLP assembly, or may be retained on the subunit and thus contained in the NP.A “variant” of a reference polypeptide sequence includes amino acid sequences having one or more amino acid substitutions, insertions and/or deletions when compared to the reference sequence. The variant may comprise an amino acid sequence which is at least 70%, at least 75%, at least 80%, at least 85%, at least 90%, at least 95%, at least 96%, at least 97%, at least 98%, or at least 99% identical to a full-length reference polypeptide.

Amino acid substitutions may be conservative substitutions. Amino acids are commonly classified into distinct groups according to their side chains. For example, some side chains are considered non-polar, i.e. hydrophobic, while some others are considered polar, i.e. hydrophilic. Alanine (A), glycine (G), valine (V), leucine (L), isoleucine (I), methionine (M), proline (P), phenylalanine (F) and tryptophan (W) are considered to be hydrophobic amino acids, while serine (S), threonine (T), asparagine (N), glutamine (Q), tyrosine (Y), cysteine (C), lysine (K), arginine (R), histidine (H), aspartic acid (D) and glutamic acid (E) are considered to be polar amino acids. Regardless of their hydrophobicity, amino acids are also classified into subgroups based on common properties shared by their side chains. For example, phenylalanine, tryptophan and tyrosine are jointly classified as aromatic amino acids and will be considered as aromatic amino acids within the meaning of the present invention. Aspartate (D) and glutamate (E) are among the acidic or negatively charged amino acids, while lysine (K), arginine (R) and histidine (H) are among the basic or positively charged amino acids, and they will be considered as such in the sense of the present invention. Hydrophobicity scales are available which utilize the hydrophobic and hydrophilic properties of each of the 20 amino acids and allocate a hydrophobic score to each amino acid, creating thus a hydrophobicity ranking As an illustrative example only, the Kyte and Dolittle scale may be used (Kyte et al. (1982)). This scale allows one skilled in the art to calculate the average hydrophobicity within a segment of predetermined length.

NP polypeptides may be modified to introduce amino acid residues known in the art as capable of being chemically conjugated to a heterologous molecule, such as an antigenic bacterial antigen, such as a bacterial polypeptide, a bacterial polysaccharide, a bacterial oligosaccharide, or a bacterial glycoconjugate; for example a GBS polypeptide, GBS polysaccharide, GBS oligosaccharide, or GBS glycoconjugate.

According to the present invention, two proteins having a high degree of identity have amino acid sequences at least 80% identical, at least 85% identical, at least 87% identical, at least 90% identical, at least 92% identical, at least 94% identical, at least 96% identical, at least 98% identical or at least 99% identical. It will be understood by those of skill in the art that the similarity between two polypeptide sequences (or polynucleotide sequences), can be expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two polypeptide (or polynucleotide) sequences, the more similar are the higher order structures resulting from folding and assembly. Methods of determining sequence identity are well known in the art. Various programs and alignment algorithms are described in: Smith and Waterman (1981); Needleman and Wunsch (1970); Higgins and Sharp (1988); Higgins and Sharp (1989); Corpet et al. (1988); and Pearson and Lipman (1988). Altschul et al. (1994) presents a detailed consideration of sequence alignment methods and homology calculations. The NCBI Basic Local Alignment Search Tool (BLAST) (Altschul et al. (1990)) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Sequence identity between polypeptide sequences is preferably determined by pairwise alignment algorithm using the Needleman-Wunsch global alignment algorithm (Needleman and Wunsch (1970)), using default parameters (e.g. with Gap opening penalty=10.0, and with Gap extension penalty=0.5, using the EBLOSUM62 scoring matrix). This algorithm is conveniently implemented in the needle tool in the EMBOSS package (Rice et al., (2000)). Sequence identity should be calculated over the entire length of polypeptide sequences.

### Expression Methods

The NP subunit polypeptides used in the present invention may be produced any suitable means, including by recombinant expression or by chemical synthesis, and purified (if necessary) using any suitable method as is known in the art. The NP product may be analyzed using methods known in the art, e.g., by crystallography, Dynamic Light Scattering (DLS), Nano-Differential Scanning Fluorimetry (Nano-DSF), and Electron Microscopy, to confirm production of suitable nanoparticles.

Methods of recombinant expression suitable for the production of the NP subunit polypeptides are known in the art. The expressed polypeptide may include a purification tag. Various expression systems are known in the art, including those using human (e g , HeLa) host cells, mammalian (e g , Chinese Hamster Ovary (CHO)) host cells, prokaryotic host cells (e.g., E. coli), or insect host cells. The host cell is typically transformed with the recombinant nucleic acid sequence encoding the desired polypeptide product, cultured under conditions suitable for expression of the product, and the product purified from the cell or culture medium. Cell culture conditions are particular to the cell type and expression vector, as is known in the art.

Host cells can be cultured in conventional nutrient media modified as appropriate and as will be apparent to those skilled in the art (e.g., for activating promoters). Culture conditions, such as temperature, pH and the like, may be determined using knowledge in the art, see e.g., Freshney (1994) and the references cited therein. In bacterial host cell systems, a number of expression vectors are available including, but not limited to, multifunctional E. coli cloning and expression vectors such as BLUESCRIPT (Stratagene) or pET vectors (Novagen, Madison Wis.). In mammalian host cell systems, a number of expression systems, including both plasmids and viral-based systems, are available commercially.

Eukaryotic or microbial host cells expressing NP subunit polypeptides can be disrupted by any convenient method (including freeze-thaw cycling, sonication, mechanical disruption), and polypeptides and/or self-assembled NPs can be recovered and purified from recombinant cell culture by any suitable method known in the art (including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxyapatite chromatography, and lectin chromatography). High performance liquid chromatography (HPLC) can be employed in the final purification steps.

In general, and using methods as are known in the art, expression of a recombinantly encoded NP subunit polypeptide involves preparation of an expression vector comprising a recombinant polynucleotide under the control of one or more promoters, such that the promoter stimulates transcription of the polynucleotide and promotes expression of the encoded polypeptide. “Recombinant Expression” as used herein refers to such a method.

“Recombinant expression vectors” comprise a recombinant nucleic acid sequence operatively linked to control sequences capable of effecting expression of the gene product. “Control sequences” are nucleic acid sequences capable of effecting the expression of the nucleic acid molecules and need not be contiguous with the nucleic acid sequences, so long as they function to direct the expression thereof. “Recombinant host cells” comprise such recombinant expression vectors.

### Purification

The term “purified” as used herein refers to the separation or isolation of a defined product (e.g., a recombinantly expressed polypeptide) from a composition containing other components (e.g., a host cell or host cell medium). A polypeptide composition that has been fractionated to remove undesired components, and which composition retains its biological activity, is considered purified. A purified polypeptide retains its biological activity. Purified is a relative term and does not require that the desired product be separated from all traces of other components. Stated another way, “purification” or “purifying” refers to the process of removing undesired components from a composition or host cell or culture. Various methods for use in purifying polypeptides and NPs are known in the art, e.g., centrifugation, dialysis, chromatography, gel electrophoresis, affinity purification, filtration, precipitation, antibody capture, and combinations thereof. Polypeptides NPs may be expressed with a tag operable for affinity purification, such as a 6×Histidine tag as is known in the art. A His-tagged polypeptide may be purified using, for example, Ni-NTA column chromatography or using anti-6×His antibody fused to a solid support.

Thus, the term “purified” does not require absolute purity; rather, it is intended as a relative term. A “substantially pure” preparation of polypeptides (or nanoparticles) or nucleic acid molecules is one in which the desired component represents at least 50% of the total polypeptide (or nucleic acid) content of the preparation. In certain embodiments, a substantially pure preparation will contain at least 60%, at least 70%, at least 80%, at least 85%, at least 90%, at least 95%, at least 97%, at least 98%, or at least 99% or more of the total polypeptide (or nucleic acid) content of the preparation. Methods for quantifying the degree of purification of expressed polypeptides are known in the art and include, for example, determining the specific activity of an active fraction, or assessing the number of polypeptides within a fraction by SDS/PAGE analysis.

### Antigenic Display

Molecules, including antigenic molecules, attached to the exterior surface of an NP of the present invention may be referred to herein as “display” or “displayed” molecules. Antigen-displaying nanoparticles preferably display multiple copies of antigenic molecules in an ordered array. It is theorized that an ordered multiplicity of antigens presented on a NP allows multiple binding events to occur simultaneously between the NP and a host's cells, which favors the induction of a potent host immune response. See e.g., Lopez-Sagaseta et al., (2016).

Presentation of antigens on NP has been exploited to improve the immunogenicity of subunit protein antigens (Jardine et al, (2013); Correira et al, (2014)). In particular, Qβ nanoparticles have been used as a scaffold for a variety of haptens (including nicotinamide/alzheimer peptides/angiotensin) (Lopez-Sagaseta et al., (2015)). QBeta nanoparticles are also known to behave as scaffolds for short synthetic cancer (Wu et al., (2019)) or bacterial (Polonskaya et al, (2017)) carbohydrates antigens. However, the impact of conjugation of medium-long carbohydrates (medium length oligosaccharides and long length polysaccharides per se exposing multiple carbohydrate epitopes in sequence) on the onset of the elicited immune response is not predictable and has never been thoroughly explored.

### Conjugation

Displayed molecules may be incorporated onto or attached to the NPs of the present invention by any suitable means.

Chemical conjugation: Functional groups present on the NP subunit polypeptides can be used for conjugation of display molecules Amino acid side-chain groups used for conjugation include amino group on lysine, thiol on cysteine, carboxylic acid on aspartic acids and glutamic acids, hydroxyl moiety on tyrosine, guanidyl moiety on arginine, imidazole moiety on histidine and indoyl moiety on tryptophan, with different chemistries known in the art. Homo- or hetero-bifunctional crosslinkers are available for conjugation. The side-chain amino groups of lysine residues are nucleophiles, so lysine residues exposed at the NP surface have large solvent accessibility and can be used as sites for conjugation to display molecules.

One or more selected amino acid residues within a subunit polypeptide sequence may be modified using methods known in the art to provide a site suitable for chemical conjugation at the NP exterior surface, where such modification does not disrupt the polypeptide activity.

One embodiment of the present invention is an NP, where one or more display molecules are chemically conjugated to lysine residues present at the exterior surface of the NP. The display molecule(s) may be a bacterial antigen, such as a bacterial polypeptide, a bacterial polysaccharide, a bacterial oligosaccharide, or a bacterial glycoconjugate; for example a GBS oligosaccharide, GBS polysaccharide, GBS glycan, or GBS glycoconjugate, or combinations thereof.

Covalent conjugation of saccharides to monomeric carrier proteins enhances the immunogenicity of saccharides as it converts them from T-independent antigens to

T-dependent antigens, thus allowing priming for immunological memory. Conjugation is particularly useful for pediatric vaccines (Ramsay et al. (2001)) and is a well-known technique (Lindberg (1999); Buttery & Moxon (2000); Ahmad & Chapnick (1999); Goldblatt (1998); European Patent 0477508; U.S. Pat. No. 5,306,492; WO98/42721; Dick et al. in Conjugate Vaccines (1989); Hermanson, (1996)).

Conjugation of bacterial saccharides, such as GBS saccharides, to monomeric carrier proteins has been widely reported (Paoletti et al. (1990)). Therefore, as used herein, the term “monomeric carrier protein” or “carrier protein” refers to an immunogenic protein which, when conjugated to a polysaccharide (or oligosaccharide) and administered to an animal, will enhance an immune response in the animal, particularly the production of antibodies that bind specifically to the conjugated polysaccharide or oligosaccharide. The typical prior art process for production of bacterial glycoconjugates, such as GBS glycoconjugates, typically involves reductive amination of a purified saccharide to a monomeric carrier protein such as tetanus toxoid (TT) or CRM197 (Wessels et al. (1990)). The reductive amination involves an amine group on the side chain of an amino acid in the monomeric carrier and an aldehyde group in the saccharide. As GBS capsular saccharides do not include an aldehyde group in their natural form then this is typically generated before conjugation by oxidation (e.g. periodate oxidation) of a portion (e.g. between 5 and 40%) of the saccharide's sialic acid residues [Wessels et al. (1990); U.S. Pat. No. 4,356,170]. GBS glycoconjugate vaccines prepared in this manner have been shown to be safe and immunogenic in humans for each of GBS serotypes Ia, Ib, II, III, and V (Paoletti & Kasper (2003)). An alternative conjugation process involves the use of —NH₂ groups in the saccharide (either from de-N-acetylation, or after introduction of amines) in conjunction with bifunctional linkers, as described (WO2006/082530). A further alternative process is described in WO96/40795 and Michon et al. (2006). In this process, the free aldehydes groups of terminal 2,5-anhydro-D-mannose residues from depolymerization of type II or type III capsular saccharides by mild cleavage through de-N-acetylation/nitrosation are used for conjugation by reductive amination. In some embodiments, one or more of the conjugates in the immunogenic compositions of the present invention have been prepared in this manner

The conjugation method may rely on activation of the saccharide with cyanylate chemistry, such as with 1-cyano-4-dimethylamino pyridinium tetrafluoroborate (CDAP) to form a cyanate ester. The activated saccharide may thus be coupled directly or via a spacer (linker) group to an amino group on the protein nanoparticle. For example, the spacer could be cystamine or cysteamine to give a thiolated polysaccharide or oligosaccharide which could be coupled to the protein nanoparticle via a thioether linkage obtained after reaction with a maleimide-activated protein nanoparticle (for example using GMBS) or a holoacetylated protein nanoparticle (for example using iodoacetimide or N-succinimidyl bromoacetatebromoacetate). Optionally, the cyanate ester (optionally made by CDAP chemistry) is coupled with hexane diamine or ADH and the amino-derivatised saccharide is conjugated to the protein nanoparticle using carbodiimide (e.g. EDAC or EDC) chemistry via a carboxyl group on the protein nanoparticle. Such conjugation methods are described in PCT published application WO 93/15760 Uniformed Services University and WO 95/08348 and WO 96/29094.

Other suitable techniques use carbiinides, hydrazides, active esters, norborane, p-nitrobenzoic acid, N-hydroxysuccinimide, S-NHS, EDC, TSTU. Many are described in WO 98/42721. Conjugation may involve a carbonyl linker which may be formed by reaction of a free hydroxyl group of the saccharide with CDI (Bethell et al J. Biol. Chem. 1979, 254; 2572-4, Hearn et al J. Chromatogr. 1981. 218; 509-18) followed by reaction of with a protein to form a carbamate linkage. This may involve reduction of the anomeric terminus to a primary hydroxyl group, optional protection/deprotection of the primary hydroxyl group′ reaction of the primary hydroxyl group with CDI to form a CDI carbamate intermediate and coupling the CDI carbamate intermediate with an amino group on a protein.

Following the conjugation (the reduction reaction and optionally the capping or quenching reaction), the glycoconjugates may be purified (enriched with respect to the amount of polysaccharide- or oligosaccharide-protein conjugate) by a variety of techniques known to the skilled person. These techniques include dialysis, concentration/diafiltration operations, tangential flow filtration, ultrafiltration, precipitation/elution, column chromatography (ion exchange chromatography, multimodal ion exchange chromatography, DEAE, or hydrophobic interaction chromatography), and depth filtration. See, e.g., U.S. Pat. No. 6,146,902. In an embodiment, the glycoconjugates are purified by diafilitration or ion exchange chromatography or size exclusion chromatography.

The conjugates can also be prepared by direct reductive amination methods as described in U.S. Pat. No. 4,365,170 (Jennings) and U.S. Pat. No. 4,673,574 (Anderson). Other methods are described in EP-0-161-188, EP-208375 and EP-0-477508.

A further method involves the coupling of a cyanogen bromide (or CDAP) activated saccharide derivatised with adipic acid hydrazide (ADH) to the protein nanoparticle by Carbodiimide condensation (Chu C. et al Infect Immunity, 1983 245 256), for example using EDAC.

In an embodiment, a hydroxyl group (optionally an activated hydroxyl group for example a hydroxyl group activated by a cyanate ester) on a saccharide is linked to an amino or carboxylic group on a protein nanoparticle either directly or indirectly (through a linker). Where a linker is present, a hydroxyl group on a saccharide is optionally linked to an amino group on a linker, for example by using CDAP conjugation. A further amino group in the linker for example ADH) may be conjugated to a carboxylic acid group on a protein nanoparticle, for example by using carbodiimide chemistry, for example by using EDAC. In an embodiment, the bacterial saccharide, such as a polysaccharide or oligosaccharide, is conjugated to the linker first before the linker is conjugated to the protein nanoparticle. Alternatively the linker may be conjugated to the protein nanoparticle before conjugation to the saccharide.

In general the following types of chemical groups on a protein nanoparticle can be used for coupling / conjugation:
Carboxyl (for instance via aspartic acid or glutamic acid). In one embodiment this group is linked to amino groups on saccharides directly or to an amino group on a linker with carbodiimide chemistry e.g. with EDAC.
Amino group (for instance via lysine). In one embodiment this group is linked to carboxyl groups on saccharides directly or to a carboxyl group on a linker with carbodiimide chemistry e.g. with EDAC. In another embodiment this group is linked to hydroxyl groups activated with CDAP or CNBr on saccharides directly or to such groups on a linker; to saccharides or linkers having an aldehyde group; to saccharides or linkers having a succinimide ester group.
Sulphydryl (for instance via cysteine). In one embodiment this group is linked to a bromo or chloro acetylated saccharide or linker with maleimide chemistry. In one embodiment this group is activated/modified with bis diazobenzidine.
Hydroxyl group (for instance via tyrosine). In one embodiment this group is activated/modified with bis diazobenzidine.
Imidazolyl group (for instance via histidine). In one embodiment this group is activated/modified with bis diazobenzidine.
Guanidyl group (for instance via arginine).
Indolyl group (for instance via tryptophan).

On a saccharide, in general the following groups can be used for a coupling: OH, COOH or NH2. Aldehyde groups can be generated after different treatments known in the art such as: periodate, acid hydrolysis, hydrogen peroxide, etc.

Direct coupling approaches include, but are not limited to:
Saccharide-OH+CNBr or CDAP→cyanate ester+NH2-Prot→conjugate
Saccharide-aldehyde+NH2-Prot→Schiff base+NaCNBH3→conjugate
Saccharide-COOH+NH2-Prot+EDAC→conjugate
Saccharide-NH2+COOH-Prot+EDAC→conjugate

Indirect coupling via spacer (linker) approaches include, but are not limited to:
Saccharide-OH+CNBr or CDAP→cyanate ester+NH2—NH2→saccharide—NH2+COOH-Prot+EDAC→conjugate
Saccharide-OH+CNBr or CDAP→cyanate ester+NH2—SH→saccharide—SH+SH-Prot (native Protein with an exposed cysteine or obtained after modification of amino groups of the protein by SPDP for instance)→saccharide-S—S-Prot
Saccharide-OH+CNBr or CDAP→cyanate ester+NH2—SH→saccharide—SH+maleimide-Prot (modification of amino groups)→conjugate
Saccharide-COOH+EDAC+NH2—NH2→saccharide—NH2+EDAC+COOH-Prot→conjugate
Saccharide-COOH+EDAC+NH2—SH→saccharide—SH+SH-Prot (native Protein with an exposed cysteine or obtained after modification of amino groups of the protein by SPDP for instance)→saccharide-S—S-Prot
Saccharide-COOH+EDAC+NH2—SH→saccharide—SH+maleimide-Prot (modification of amino groups)→conjugate
Saccharide-Aldehyde+NH2—NH2→saccharide—NH2+EDAC+COOH-Prot→conjugate

### Antigens

One embodiment of the present invention is a nanoparticle displaying one or more bacterial capsular polysaccharide or oligosaccharide antigens, such as GBS poly- or oligosaccharide antigens, or GBS glycoconjugates, on the exterior surface of the nanoparticle.

In one embodiment of the present invention the bacterial capsular polysaccharide or oligosaccharide antigens, such as GBS poly- or oligosaccharide antigens, displayed on the exterior surface of the nanoparticle are not conjugated to a carrier protein, such as TT, DT or CRM197. Stated another way, the bacterial capsular polysaccharide or oligosaccharide antigens, such as GBS poly- or oligosaccharide antigens, are conjugated to polypeptides that make up the NP but are not conjugated to any other polypeptide.

In one embodiment of the present invention, the antigen displayed on the NP is a GBS capsular polysaccharide or oligosaccharide, or immunogenic fragment thereof, or combinations of such. The GBS capsular polysaccharide or oligosaccharide may be selected from any serotype, including Ia, Ib, II, III, IV and V. A single NP may display polysaccharides or oligosaccharides, or immunogenic fragments thereofc, from more than one bacterial serotype, such as more than one GBS serotype.

In a further embodiment of the present invention, the antigen displayed on the NP is a polysaccharide or oligosaccharide antigen from a bacterial species selected from the group consisting of a Acinetobacter species, Bacillus species, Bordetella species, Borrelia species, Burkholderia species, Campylobacter species, Candida species, Chlamydia species, Clostridium species, Corynebacterium species, Enterococcus species, Escherichia species, Francisella species, Haemophilus species, Helicobacter species, Klebsiella species, Legionella species, Listeria species, Neisseria species, Proteus species, Pseudomonas species, Salmonella species, Shigella species, Staphylococcus species, Streptococcus species, Streptomyces species, Vibrio species, and Yersinia species.

Methods of producing NPs

A further embodiment of the invention is a method of producing an NP comprising bacterial capsular polysaccharide or oligosaccharide antigens, such as GBS poly- or oligosaccharide antigens, on the exterior surface of the NP. The method comprises one or more of the steps of (a) culturing a recombinant host cell expressing the NP subunit polypeptide(s) invention under conditions conducive to the expression of the polypeptide and self-assembly of the NP; (b) recovering or purifying assembled NPs from the host cell or the culture medium in which the host cell is grown, as is suitable; (c) extracting and purifying native polysaccharide from bacteria, such as GBS bacteria, (d) optionally preparing bacterial oligosaccharides, such as GBS oligosaccharides, either by chemical or enzymatic depolymerization or synthetic approach and (e) conjugating optionally derivatized bacterial polysaccharide or oligosaccharide antigen, such as GBS polysaccharide or oligosaccharide antigen, to the exterior of the NP.

### Compositions

A further embodiment of the present invention is immunogenic compositions or pharmaceutical compositions, such as vaccines, which comprise NPs displaying bacterial polysaccharide or oligosaccharide antigens, such as GBS oligo- or polysaccharide antigens, and a pharmaceutically acceptable diluent, or excipient. In certain instances, immunogenic compositions are administered to subjects to elicit an immune response that protects the subject against infection by a pathogen, or decreases symptoms or conditions induced by a pathogen. In the context of this disclosure, the term immunogenic composition will be understood to encompass compositions that are intended for administration to a subject or population of subjects for the purpose of eliciting a protective or palliative immune response against a bacterial pathogen, such as a Acinetobacter species, Bacillus species, Bordetella species, Borrelia species, Burkholderia species, Campylobacter species, Candida species, Chlamydia species, Clostridium species, Corynebacterium species, Enterococcus species, Escherichia species, Francisella species, Haemophilus species, Helicobacter species, Klebsiella species, Legionella species, Listeria species, Neisseria species, Proteus species, Pseudomonas species, Salmonella species, Shigella species, Staphylococcus species, Streptococcus species, Streptomyces species, Vibrio species, and Yersinia species.

An “immunogenic composition” is a composition of matter suitable for administration to a human or non-human mammalian subject and which, upon administration of an immunologically effective amount, elicits a specific immune response, e.g., against an antigen displayed on the NP. An immunogenic composition of the present invention can include one or more additional components, such as an excipient, and/or adjuvant. While administration of an antigen displayed on NPs may enhance a subject's immune response to the antigen (compared to administration of the antigen in the absence of the NP), as used herein, the nanoparticle scaffolds are not defined as an adjuvant.

Numerous pharmaceutically acceptable diluents and/or pharmaceutically acceptable excipients are known in the art and are described, e.g., in Remington's Pharmaceutical Sciences, by E. W. Martin, Mack Publishing Co., Easton, PA, 15th Edition (1975). The adjective “pharmaceutically acceptable” indicates that the diluent or excipient is suitable for administration to a subject (e.g., a human or non-human mammalian subject). In general, the nature of the diluent and/or excipient will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. In certain formulations (for example, solid compositions, such as powder forms), a liquid diluent is not employed. In such formulations, non-toxic solid components can be used, including for example, pharmaceutical grades of trehalose, mannitol, lactose, starch or magnesium stearate. Suitable solid components are typically large, slowly metabolized macromolecules such as proteins (e.g., nanoparticles), polysaccharides, polylactic acids, polyglycolic acids, polymeric amino acids, amino acid copolymers, lipid aggregates (such as oil droplets or liposomes), and inactive virus particles.

Accordingly, suitable excipients can be selected by those of skill in the art to produce a formulation suitable for delivery to a subject by a selected route of administration.

Immunogenic compositions of the present invention may additionally include one or more adjuvants. An “adjuvant” is an agent that enhances the production of an immune response in a non-specific manner Common adjuvants include suspensions of minerals (alum, aluminum hydroxide, aluminum phosphate); saponins such as QS21; emulsions, including water-in-oil, and oil-in-water (and variants thereof, including double emulsions and reversible emulsions), liposaccharides, lipopolysaccharides, immunostimulatory nucleic acid molecules (such as CpG oligonucleotides), liposomes, Toll Receptor agonists, Toll-like Receptor agonists (particularly, TLR2, TLR4, TLR7/8 and TLR9 agonists), and various combinations of such components. For the purposes of the present invention, the NP or VLP is not considered an adjuvant.

In one embodiment of the present invention, the immunogenic or pharmaceutical compositions comprising NPs of the present invention do not further comprise an adjuvant.

Preparation of immunogenic compositions, such as vaccines, including those for administration to human subjects, is generally described in Pharmaceutical Biotechnology, Vol.61 Vaccine Design-the subunit and adjuvant approach, edited by Powell and Newman, Plenum Press, 1995. See also New Trends and Developments in Vaccines, edited by Voller et al., University Park Press, Baltimore, Maryland, U.S.A. 1978.

### Prophylactic and Therapeutic Uses

Bacterial infections have a large impact on public health. For example, GBS is a major cause of neonatal sepsis and meningitis in infants born to women carrying the bacteria. At birth, a neonate's immune system is still developing, and they are vulnerable to infection by vertically acquired and postnatally acquired GBS. Immunization of a female subject to produce antibodies that can, during pregnancy, be passively transferred across the placenta to a gestating infant is referred to herein as maternal immunization, maternal vaccination, or as a maternally administered vaccine. See, e.g., Englund, 2007. Maternal immunization has been previously investigated using saccharide based vaccines, including meningococcal vaccines (see, e.g.,Shahid et al. 2002; Quimbao et al. 2007; O'Dempsey et al. 1996).

In women who have not received a GBS vaccine, an inverse relationship has been reported between levels of naturally occurring GBS serotype-specific IgG antibodies at the time of delivery and the risk of neonatal infection. See e.g., Lin et al. (2001), Lin et al. (2004), Baker et al. (2014), Dangor et al. (2015) and Fabbrini et al. (2016). Lin et al. (2001) report that neonates born to women who had levels of IgG GBS Ia antibody≥5 μg/mL had an 88% lower risk (95% confidence interval, 7%-98%) of developing type-specific EOD, compared with neonates born to women who had levels<0.5 μg/mL. Baker et al. (2014) estimated that the absolute risk of a neonate contracting GBS EOD due to serotypes Ia, III and V would decrease by 70% if maternal CPS-specific antibody concentrations were equal or higher than 1 Fabbrini et al., (2016) reported that maternal anti-capsular IgG concentrations above 1 μg/mL mediated GBS killing in vitro and were predicted to respectively reduce by 81% and 78% the risk of GBS Ia and III early-onset disease in Europe. Dangor et al. (2015) report that the risk of neonatal invasive GBS disease was less than 10% when maternal antibody concentrations were≥6 μg/mL and ≥3 μg/mL for serotypes Ia and III, respectively. However, as noted in Kobayashi et al. (2016), it is unclear the extent to which correlates of protection may be inferred from the evaluation of natural immunity in observational studies.

In some prior studies of maternal immunization against GBS, a boosting dose was administered one month (30 days) after the priming dose. See Madhi et al. (2016), Leroux-Roel et al. (2016). WO 2018/229708 reports that an extended period (more than 30 days) between prime and boost was beneficial in eliciting GBS serotype-specific maternal antibodies that could be transferred to a gestational infant, and that IgG titers in maternal sera from vaccinated women were predictive of opsonophagocytic killing assay (OPKA) titers against GBS serotypes, indicating comparable functional activity of naturally-acquired and vaccine-induced GBS antibodies. In the study reported in Donders et al. (2016), more than 50% of women (Belgium and Canada) in both the vaccine and placebo groups had baseline GBS antibody concentrations below the lower limit of quantification (LLOQ) for Ia, Ib, and III serotypes. After vaccination, antibody GMCs were statistically higher for women who were at or above the LLOQ at baseline, compared with those below the LLOQ at baseline. Similarly, Heyderman (2016) reported undetectable antibody concentrations at baseline (<LLOQ) for about 69-80% of women against serotype Ia, 1-6% of women against serotype Ib, and 34-43% of women against serotype III. Antibody GMCs post-vaccination were higher in subjects who had baseline antibody concentrations>LLOQ.

For effective vaccination of pregnant woman against GBS and other bacterial pathogens, a vaccine capable of eliciting a strong antibody response with a single dose in subjects who are seronegative at baseline is desirable.

A further aspect of the present invention is a method of inducing an immune response in a mammalian subject, such as a human subject, wherein said immune response is specific for a bacterial antigenic molecule, such as a bacterial polysaccharide or oligosaccharide antigen, displayed on the surface of NPs of the present invention. The method comprises administering to a subject an immunologically effective amount of a NP displaying the bacterial antigenic molecule to which an immune response is desired. The subject may have a bacterial infection at the time of administration, or the administration may be given prophylactically to a subject who does not have a bacterial infection at the time of administration.

In one embodiment, the NP administered displays at least one bacterial antigenic molecule, such as a bacterial saccharide (such as a polysaccharide or oligosaccharide), selected from at least one pathogenic bacterial species. In some embodiments the antigenic molecule is a bacterial saccharide, such as polysaccharide or oligosaccharide. The bacterial saccharide may be a capsular saccharide or O-antigen saccharide. The bacterial saccharide, such as polysaccharide or oligosaccharide, may be selected from a bacterial species selected from the group consisting of a Acinetobacter species, Bacillus species, Bordetella species, Borrelia species, Burkholderia species, Campylobacter species, Candida species, Chlamydia species, Clostridium species, Corynebacterium species, Enterococcus species, Escherichia species, Francisella species, Haemophilus species, Helicobacter species, Klebsiella species, Legionella species, Listeria species, Neisseria species, Proteus species, Pseudomonas species, Salmonella species, Shigella species, Staphylococcus species, Streptococcus species, Streptomyces species, Vibrio species, and Yersinia species.

In one embodiment, the NP administered displays bacterial saccharide antigens from at least two (i.e., two or more) pathogenic bacterial species or serotypes. This may be achieved by administering a mixture of NP where each NP displays a bacterial saccharide antigen from a single bacterial species or serotype, or by administering a NP that display bacterial saccharides from multiple (such as two, three, four, five or more) species or serotypes.

In one embodiment, the NP administered displays GBS CPS antigens from at least two disease-causing GBS serotypes, such as from any of serotypes Ia, Ib, II, III, IV, V, VI, VII, VIII, and IX. This may be achieved by administering a mixture of NPs where each NP displays a single GBS serotype antigen, or by administering NPs that display multiple GBS serotype antigens. The GBS antigens may be capsular polysaccharides or immunogenic fragments thereof, oligosaccharides, GBS glycoconjugates, or a mixture thereof.

A further aspect of the present invention is a method of inducing an immune response for the purpose of preventing and/or treating a bacterial infection in a subject, comprising administering to the subject an immunologically effective amount of the NPs of the present invention that display at least one bacterial antigenic molecule to which an immune response is desired, wherein said at least one antigen can induce a protective or therapeutic immune response. Such NPs may be within an immunogenic or pharmaceutical composition as described herein. In a specific embodiment, the administration is to a pregnant human subject, or one intending to become pregnant, and the method is to prevent a bacterial infection in an infant born to the subject by transplacental transfer of maternal antibodies. In one embodiment of the invention, a single dose is administered to the subject. The dose may be adjuvant-free, or it may further comprise an adjuvant.

A further aspect of the present invention is a method of inducing an immune response for the purpose of treating and/or preventing a GBS infection of a subject, comprising administering to the subject an immunologically effective amount of the NPs of the present invention that display the GBS antigenic molecule to which an immune response is desired, wherein said antigens can induce a protective or therapeutic immune response. Such NPs may be within an immunogenic or pharmaceutical composition as described herein. In a specific embodiment, the administration is to a pregnant human subject, or one intending to become pregnant, and the method is to prevent GBS infection in an infant born to the subject by transplacental transfer of maternal antibodies.

In one embodiment, a single dose of the NP displaying the bacterial antigenic molecule is capable of inducing a protective or therapeutic immune response to bacterial infection. In another embodiment of the invention, a single dose is administered to the subject. In another embodiment, two doses are administered to the subject with an interval of at least 1 year, at least 2 years, at least 3 years, at least 4 years or at least 5 years between doses. The dose may be adjuvant-free, or it may further comprise an adjuvant.

Another embodiment of the present invention is a method of immunising a human female subject in order to decrease the risk of Group B Streptococcus (GBS) disease in an infant born to the subject, where the female receives both a priming dose and a boosting dose of a composition according to the present invention, and where the priming and the boosting dose each elicit in the subject IgG antibodies specific for the same disease-causing Group B Streptococcus serotype(s). In one embodiment, the boosting dose is administered more than thirty days after the priming dose. In one embodiment, GBS antigen component of the priming and/or the boosting dose comprises GBS CPS antigens from at least two disease-causing GBS serotypes, such as selected from serotypes Ia, Ib, II, III, IV, V, VI, VII, VIII, and IX. The priming and/or boosting dose may be adjuvant-free, or either or both may further comprise an adjuvant. In an embodiment of the present invention, the priming dose is administered to a non-pregnant female subject, and the boosting dose is administered to the subject when pregnant.

Thus, in one embodiment, the NPs and compositions of the present invention are utilized in methods of immunizing a subject to achieve a protective (prophylactic) immune response in both the subject and (via transplacental transfer of maternal antibodies) to an infant born to the subject.

The immunogenic compositions of the invention are conventionally administered parenterally, e.g., by injection, either subcutaneously, intraperitoneally, transdermally, or intramuscularly. Dosage treatment may be a single dose schedule or a multiple dose schedule.

A further aspect of the present invention is a method of inducing an immune response in a mammalian subject, such as a human subject, wherein said immune response is specific for a bacterial antigenic molecule displayed on the surface of NPs of the present invention. The method comprises administering to a subject an immunologically effective amount of the NPs displaying the bacterial antigenic molecule to which an immune response is desired. The subject may have a bacterial infection at the time of administration, or the administration may be given prophylactically to a subject who does not have a bacterial infection at the time of administration. In one embodiment, the NPs administered display bacterial antigens from at least two disease-causing bacterial serotypes. In another embodiment, the NPs administered display bacterial antigens from at least two disease-causing bacterial species. This may be achieved by administering a mixture of NPs where each NP displays a single bacterial serotype antigen or a single bacterial species antigen, or by administering NPs that display multiple bacterial serotype antigens or multiple bacterial species antigens. The bacterial antigens may be bacterial saccharides, such as polysaccharides or oligosaccharides. The bacterial saccharides may be capsular saccharides or O-antigen saccharides, immunogenic fragments thereof, glycoconjugates, or a mixture of two or more of the foregoing. The bacterial antigens may be selected from a bacterial species selected from the group consisting of a Acinetobacter species, Bacillus species, Bordetella species, Borrelia species, Burkholderia species, Campylobacter species, Candida species, Chlamydia species, Clostridium species, Corynebacterium species, Enterococcus species, Escherichia species, Francisella species, Haemophilus species, Helicobacter species, Klebsiella species, Legionella species, Listeria species, Neisseria species, Proteus species, Pseudomonas species, Salmonella species, Shigella species, Staphylococcus species, Streptococcus species, Streptomyces species, Vibrio species, and Yersinia species Yersinia.

A further aspect of the present invention is a method of inducing an immune response for the purpose of treating and/or preventing a bacterial infection of a subject, comprising administering to the subject an immunologically effective amount of the NPs of the present invention that display the bacterial antigenic molecule to which an immune response is desired, wherein said antigens can induce a protective or therapeutic immune response. Such NPs may be within an immunogenic or pharmaceutical composition as described herein. In a specific embodiment, the administration is to a pregnant human subject, or one intending to become pregnant, and the method is to prevent bacterial infection in an infant born to the subject by transplacental transfer of maternal antibodies. In one embodiment of the invention, a single dose is administered to the subject. The dose may be adjuvant-free, or it may further comprise an adjuvant.

Another embodiment of the present invention is a method of immunising a human subject, where the subject receives both a priming dose and a boosting dose of a composition according to the present invention, and where the priming and the boosting dose each elicit in the subject IgG antibodies specific for the same disease-causing bacterial serotype(s). In one embodiment, the boosting dose is administered more than thirty days after the priming dose. In one embodiment, the bacterial antigen component of the priming and/or the boosting dose comprises bacterial CPS antigens from at least two disease-causing bacterial serotypes. The priming and/or boosting dose may be adjuvant-free, or either or both may further comprise an adjuvant.

Another embodiment of the present invention is a method of immunising a human female subject in order to decrease the risk of bacterial infection in an infant born to the subject, where the female receives both a priming dose and a boosting dose of a composition according to the present invention, and where the priming and the boosting dose each elicit in the subject IgG antibodies specific for the same disease-causing bacterial serotype(s). In one embodiment, the boosting dose is administered more than thirty days after the priming dose. In one embodiment, the bacterial antigen component of the priming and/or the boosting dose comprises bacterial CPS antigens from at least two disease-causing bacterial serotypes. The priming and/or boosting dose may be adjuvant-free, or either or both may further comprise an adjuvant. In an embodiment of the present invention, the priming dose is administered to a non-pregnant female subject, and the boosting dose is administered to the subject when pregnant.

The various features which are referred to in individual sections above apply, as appropriate, to other sections. Consequently, features specified in one section may be combined with features specified in other sections, as appropriate. Those skilled in the art will recognize or be able to ascertain using no more than routine experimentation, many equivalents to the specific embodiments of the invention (or aspects of the disclosure) described herein. Embodiments of the invention include:
C1. A protein nanoparticle having an antigenic molecule conjugated to its exterior surface, wherein the antigenic molecule is a bacterial saccharide.
C2. The protein nanoparticle of C1 wherein the bacterial saccharide is a polysaccharide or an oligosaccharide.
C3. The protein nanoparticle of any one of C1 to C2 wherein the bacterial saccharide is a capsular saccharide or O-antigen saccharide.
C4. The protein nanoparticle of any one of C1 to C3 wherein the bacterial saccharide is from a bacterial species selected from the group consisting of a Acinetobacter species, Bacillus species, Bordetella species, Borrelia species, Burkholderia species, Campylobacter species, Candida species, Chlamydia species, Clostridium species, Corynebacterium species, Enterococcus species, Escherichia species, Francisella species, Haemophilus species, Helicobacter species, Klebsiella species, Legionella species, Listeria species, Neisseria species, Proteus species, Pseudomonas species, Salmonella species, Shigella species, Staphylococcus species, Streptococcus species, Streptomyces species, Vibrio species, and Yersinia species.
C5. The protein nanoparticle of any one of C1 to C4 wherein the bacterial saccharide is from a Streptococcus species selected from Streptococcus agalactiae (Group B Streptococcus, or GBS) and Streptococcus pneumoniae.
C6. The protein nanoparticle of any one of C1 to C5 wherein said bacterial saccharide is from a GBS serotype selected from serotypes Ia, Ib, II, III, IV, V, VI, VII, VIII, and IX.
C7. The protein nanoparticle of any one of C1 to C5 wherein said bacterial saccharide is from a Streptococcus pneumoniae serotype selected from serotypes 1, 2, 3, 4, 5, 6A, 6B, 7F, 8, 9N, 9V, 10A, 11A, 12F, 14, 15B, 17F, 18C, 19A, 19F, 20, 23F, 33F.
C8. The protein nanoparticle of any one of C1 to C7 wherein said protein nanoparticle is conjugated to bacterial saccharides from at least two bacterial species or serotypes.
C9. The protein nanoparticle of any one of C1 to C8 wherein the bacterial saccharide is not conjugated to a monomeric carrier protein.
C10. The protein nanoparticle of any one of C1 to C9 wherein the bacterial saccharide is conjugated to an amino acid selected from the group consisting of a lysine residue, a cysteine residue, an aspartic acid residue, a glutamic acid residue, a tyrosine residue, an arginine residue, a histidine residue, and a tryptophan residue.
C11. The protein nanoparticle of any one of C1 to C10 wherein the bacterial saccharide is conjugated directly to the protein nanoparticle or via a spacer (linker) group.
C12. The protein nanoparticle of any one of C1 to C11 wherein the bacterial saccharide is conjugated to the protein nanoparticle by a method selected from the group consisting of (a) reductive amination; (b) carbodiimide chemistry (for example EDAC OR EDC); (c) maleimide chemistry; and (d) cyanylation chemistry (for example CDAP).
C13. The protein nanoparticle of any one of C1 to C12 wherein the bacterial saccharide is modified with a hydrazine linker, for example adipic acid dihydrazide (ADH).
C14. The protein nanoparticle of any one of C1 to C13 wherein the bacterial saccharide comprises an active ester spacer, for example SIDEA.
C15. The protein nanoparticle of any one of C1 to C14 wherein the protein nanoparticle is a non-viral protein nanoparticle or a virus-like particle (VLP).
C16. The protein nanoparticle of any one of C1 to C15 wherein the protein nanoparticle is a non-viral protein nanoparticle selected from a GBS ferritin nanoparticle or an mI3 nanoparticle.
C17. The protein nanoparticle of any one of C1 to C15 wherein the protein nanoparticle is a bacteriophage VLP.
C18. The protein nanoparticle of any one of C1 to C15 wherein the protein nanoparticle is a QBeta VLP.
C19. The protein nanoparticle of any one of C1 to C18 wherein the protein nanoparticle comprises a subunit polypeptide having at least 95%, at least 96%, at least 97%, at least 98% at least 99% or 100% sequence identity to any one of SEQ ID NO: 1, SEQ ID NO: 2, SEQ ID NO:3, SEQ ID NO: 4, SEQ ID NO: 5, SEQ ID NO: 6, SEQ ID NO: 7, or SEQ ID NO: 11, wherein the subunit protein is capable of self-assembling to form the nanoparticle.
C20. The protein nanoparticle of any one of C1 to C19 wherein the protein nanoparticle is selected from the group consisting of: (a) a QBeta VLP having a GBS saccharide conjugated to its external surface; (b) a QBeta VLP having a Streptococcus pneumoniae saccharide conjugated to its external surface; (c) a GBS ferritin nanoparticle having a GBS saccharide conjugated to its external surface; (d) a GBS ferritin nanoparticle having a Streptococcus pneumoniae saccharide conjugated to its external surface; (e) an mI3 nanoparticle having a GBS saccharide conjugated to its external surface; (f) an mI3 nanoparticle having a Streptococcus pneumoniae saccharide conjugated to its external surface.
C21. The protein nanoparticle of any one of C1 to C20, wherein said nanoparticle is capable of eliciting a protective immune response in a subject following a single dose.
C22. The protein nanoparticle of any one of C1 to C21, wherein said nanoparticle is capable of eliciting a higher immune response to the bacterial saccharide after one dose compared to after one dose of a monomeric protein carrier, such as CRM197, conjugated to the same bacterial saccharide.
C23. The protein nanoparticle of any one of C1 to C22, wherein said nanoparticle is capable of eliciting a higher or comparable immune response to the bacterial saccharide after one dose compared to after two doses of a monomeric protein carrier, such as CRM197, conjugated to the same bacterial saccharide.
C24. An immunogenic composition comprising at least one protein nanoparticle according to any one of C1-C23.
C25. The immunogenic composition of C24, wherein said composition comprises at least two nanoparticles, wherein each nanoparticle is conjugated to a different bacterial saccharide.
C26. The immunogenic composition of C24 or C25, further comprising an adjuvant.
C27. The immunogenic composition according to any one of C24 to C26, wherein said adjuvant is selected from the group consisting of alum, aluminum hydroxide, aluminum phosphate, a saponin, a water-in-oil emulsion, an oil-in-water emulsion, a liposaccharide, a lipopolysaccharide, an immunostimulatory nucleic acid molecules, a liposome, and a Toll Receptor or Toll-Like Receptor agonist.
C28. The immunogenic composition according to C24 or C25, which does not further comprise an adjuvant.
C29. The immunogenic composition according to any one of C24 to C28, which does not comprise CRM197, Diphtheria Toxoid (DT), or Tetanus Toxoid (TT).
C30. A method of producing the protein nanoparticle of any one of C1 to C23, comprising one or more of the steps of (a) culturing a recombinant host cell expressing the NP subunit polypeptide(s) of the invention under conditions conducive to the expression of the polypeptide(s) and self-assembly of the NP; (b) recovering or purifying assembled NPs from the host cell or the culture medium in which the host cell is grown, as is suitable; (c) extracting and purifying native polysaccharide from bacteria, (d) optionally preparing bacterial oligosaccharides, and (e) conjugating bacterial polysaccharide or oligosaccharide antigen to the exterior of the NP.
C31. The method of C30, further comprising the step of derivatizing the bacterial polysaccharide or oligosaccharide before step (e).
C32. The method of C30 or C31 wherein step (e) comprises conjugating the bacterial saccharide to an amino acid selected from the group consisting of a lysine residue, a cysteine residue, an aspartic acid residue, a glutamic acid residue, a tyrosine residue, an arginine residue, a histidine residue, and a tryptophan residue.
C33. The method according to any one of C30 to C32 wherein step (e) comprises conjugating the bacterial saccharide directly to the protein nanoparticle or via a spacer (linker) group.
C34. The method according to any one of C30 to C33 wherein step (e) comprises conjugating the bacterial saccharide to the protein nanoparticle by a method selected from the group consisting of (a) reductive amination; (b) carbodiimide chemistry (for example EDAC OR EDC); (c) maleimide chemistry; and (d) cyanylation chemistry (for example CDAP).
C35. The method according to any one of C30 to C34, wherein the bacterial saccharide is modified with a hydrazine linker, for example adipic acid dihydrazide (ADH).
C36. The method according to any one of C30 to C35 wherein the bacterial saccharide comprises an active ester spacer, for example SIDEA.
C37. The protein nanoparticle according to any one of C1 to C23 or the immunogenic composition according to any one of C24 to C29, for use in the prevention and/or treatment of a bacterial infection in a human subject.

YersiniaC38. Use of the protein nanoparticle according to any one of C1 to C23 or the immunogenic composition according to any one of C24 to C29 for the manufacture of a medicament for inducing an immune response in a human subject.
C39. Use of the protein nanoparticle according to any one of C1 to C23 or the immunogenic composition according to any one of C24 to C29 in the prevention or treatment of disease in a human subject.
C40. Use of the protein nanoparticle according to any one of C1 to C23 or the immunogenic composition according to any one of C24 to C29 in the prevention or treatment of bacterial infection in a human subject.
C41. Use of the protein nanoparticle according to any one of C1 to C23 or the immunogenic composition according to any one of C24 to C29 for inducing an immune response in a subject.
C42. A method of inducing an immune response in a human subject, comprising administering to the subject an immunologically effective amount of the protein nanoparticle according to any one of C1 to C23 or the immunogenic composition according to any one of C24 to C29.
C43. A method of preventing or treating a bacterial infection in a human subject, comprising administering to the subject an immunologically effective amount of the protein nanoparticle according to any one of C1 to C23 or the immunogenic composition according to any one of C24 to C29.
C44. The use according to any one of C38 to C41 or the method according to C42 or C43, wherein said subject receives a single administration of said protein nanoparticle or said immunogenic composition.
C45. The use according to any one of C38 to C41 or the method according to C42 or C43, wherein said subject receives an intramuscular administration.
C46. The use according to any one of C38 to C41 or the method according to C42 or C43, wherein said protein nanoparticle is capable of eliciting a higher immune response to the bacterial saccharide after one dose compared to after one dose of a monomeric protein carrier, such as CRM197, conjugated to the same bacterial saccharide.
C47. The use according to any one of C38 to C41 or C44 to C46, or the method according to C42 or C43 or C44 to C46, wherein said protein nanoparticle is capable of eliciting a higher or comparable immune response to the bacterial saccharide after one dose compared to after two doses of a monomeric protein carrier, such as CRM197, conjugated to the same bacterial saccharide.

### Terms

To facilitate review of the various embodiments of this disclosure, the following explanations of terms are provided. Additional terms and explanations are provided in the context of this disclosure. Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

“Nanoparticles (NP)” as used herein refers to particles of less than about 100 nm in size (less than about 100 nm in maximum diameter for spherical, or roughly spherical, particles).

“Virus-like particles (VLPs)” are multiprotein structures that mimic the organization and conformation of authentic native viruses but lack the viral genome. VLPs are considered NPs for purposes of this disclosure. A typical embodiment of a virus-like particle in accordance with the present invention is a viral capsid of a virus or bacteriophage. The terms “viral capsid” or “capsid”, refer to a macromolecular assembly composed of viral protein subunits, such as 60, 120, 180, 240, 300, 360 or more than 360 viral protein subunits.

“Virus-like particle of an RNA bacteriophage,” as used herein, refers to a virus-like particle comprising, or preferably consisting essentially of, or consisting of, coat proteins, mutants or fragments thereof, of an RNA bacteriophage.

The term “recombinant VLP” as used herein refers to a VLP that is obtained by a process which comprises at least one step of recombinant DNA technology.

Viral “coat protein” and “capsid protein.” The term viral “coat protein” is used interchangeably herein with viral “capsid protein,” and refers to a protein, such as a subunit of a natural capsid of a virus, which is capable of being incorporated into a virus capsid or a VLP. For example, the specific gene product of the Coat Protein gene of RNA bacteriophage QBeta is referred to as “QBeta CP”, whereas the “coat proteins” or “capsid proteins” of bacteriophage QBeta comprise the QBeta CP as well as the A1 protein.

As used herein the terms “protein” and “polypeptide” are used interchangeably. A protein or polypeptide sequence refers to a contiguous sequence of two or more amino acids linked by a peptide bond. The proteins and polypeptides of the invention may comprise L-amino acids, D-amino acids, or a combination thereof.

The term “fragment,” in reference to a polypeptide (or polysaccharide or oligosaccharide) antigen, refers to a contiguous portion (that is, a subsequence) of that polypeptide (or polysaccharide). An “immunogenic fragment” of a polypeptide, polysaccharide or oligosaccharide refers to a fragment that retains at least one immunogenic epitope (e.g., a predominant immunogenic epitope or a neutralizing epitope).

As used herein, a “polypeptide subunit” of a nanoparticle, or “subunit”, refers to a polypeptide that, in combination with other polypeptide subunits, self-assembles into a nanoparticle. The subunit may further comprise a polypeptide sequence which extends from the surface of the nanoparticle (i.e., is ‘displayed’ by the nanoparticle), a purification tag, or other modifications as are known in the art and that do not interfere with the ability to self-assemble into a nanoparticle.

As used herein, a “variant” polypeptide refers to a polypeptide having an amino acid sequence which is similar, but not identical to, a reference sequence, wherein the biological activity of the variant protein is not significantly altered. Such variations in sequence can be naturally occurring variations or they can be engineered through the use of genetic engineering techniques as known to those skilled in the art. Examples of such techniques may be found, e.g., in Sambrook et al., Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6.

As used herein, a “fusion polypeptide” or “chimeric polypeptide” is a polypeptide comprising amino acid sequences from at least two unrelated proteins that have been joined together, via a peptide bond, to make a single polypeptide. The unrelated amino acid sequences can be joined directly to each other or they can be joined using a linker sequence. As used herein, polypeptides are unrelated if their amino acid sequences are not normally found joined together via a peptide bond in their natural environment(s) (e.g. ,inside a cell). For example, the amino acid sequences of monomeric subunits that make up GBS ferritin, and the amino acid sequences of GBS surface proteins, are considered unrelated.

As used herein, an “antigen” is a molecule (such as a protein or saccharide), a compound, composition, or substance that stimulates an immune response by producing antibodies and/or a T cell response in a mammal, including compositions that are injected, absorbed or otherwise introduced into a mammal The term “antigen” includes all related antigenic epitopes. The term “epitope” or “antigenic determinant” refers to a site on an antigen to which B and/or T cells respond. The “predominant antigenic epitopes” are those epitopes to which a functionally significant host immune response, e.g., an antibody response or a T-cell response, is made. Thus, with respect to a protective immune response against a pathogen, the predominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

As used herein, the term “immunogenic” refers to the ability of a specific antigen, or a specific region thereof, to elicit an immune response to that antigen or region thereof when administered to a mammalian subject. The immune response may be humoral (mediated by antibodies) or cellular (mediated by cells of the immune system), or a combination thereof.

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”), such as a GBS antigen. A “protective immune response” is an immune response that inhibits a detrimental function or activity of a pathogen, prevents infection by a pathogen in an individual, or decreases symptoms that result from infection by the pathogen. A protective immune response can be measured, for example, by measuring resistance to pathogen challenge in vivo.

A “higher” immune response means an immune response that is higher than the immune response of a reference treatment. For example, IgG titers induced by a protein nanoparticle described herein (for example, as measured by Luminex/ELISA) are considered higher than the IgG titers of a reference treatment if the IgG titers are statistically higher at a p value of 0.05 or lower (such as, for example, p≤0.05, p≤0.01, p≤0.005,or p≤0.001) when calculated by well-known methods, such as the Mann-Whitney Test. OPKA titers elicited by a nanoparticle described herein as measured in pooled sera are considered higher than a reference treatment where there is at least a 3-fold increase as compared to the reference treatment.

A “comparable” immune response means an immune response that does not meet the threshold of a higher (or lower) immune response. For example, comparable IgG titers between treatment groups would be those that are not statistically higher or lower than the immune response of a reference treatment at a p value of 0.05 or lower (such as, for example, p<0.05, p<0.01, p<0.005,or p<0.001). OPKA titers between treatment groups are considered comparable if there is less than a 3-fold difference between the groups.

An “effective amount” means an amount sufficient to cause the referenced effect or outcome. An “effective amount” can be determined empirically and in a routine manner using known techniques in relation to the stated purpose. An “immunologically effective amount” is a quantity of an immunogenic composition sufficient to elicit an immune response in a subject (either in a single dose or in a series). Commonly, the desired result is the production of an antigen (e.g., pathogen)-specific immune response that is capable of or contributes to protecting the subject against the pathogen. Obtaining a protective immune response against a pathogen can require multiple administrations of the immunogenic composition; preferably a single administration is required.

As used herein, a “glycoconjugate” is a carbohydrate moiety (such as a polysaccharide or oligosaccharide) covalently linked to a moiety that is a different chemical species, such as a protein, peptide, lipid or lipid. A “GBS glycoconjugate”, as used herein, refers to a conjugate of a GBS capsular saccharide molecule and a monomeric carrier protein molecule, including the carrier proteins TT, DT, and CRM197, but excluding a GBS capsular saccharide molecule conjugated to a polypeptide subunit of an NP, including a non-viral NP or VLP.

As used herein, where a nucleic acid sequence is operably linked to another polynucleotide molecule that it is not associated with in nature, the two sequences may be referred to as “heterologous” with regard to each other. Similarly, when a polypeptide is covalently linked to (including via a linker or intervening sequence), or is in a complex with, another protein that it is not associated with in nature, the polypeptides may be referred to as “heterologous” with regard to each other. A polypeptide (or nucleic acid) sequence that is “heterologous” to GBS refers to a polypeptide (or nucleic acid) sequence that is not found in naturally occurring GBS cells. Further, when a host cell comprises a nucleic acid molecule or polypeptide that it does not naturally comprise, the nucleic acid molecule and polypeptide may be referred to as “heterologous” to the host cell. For purposes of the present invention, in a fusion protein of two polypeptides from the same host organism (such as GBS), where the polypeptides are not naturally covalently associated with each other, the two polypeptides are considered heterologous to each other. Thus, for example, a protein comprising a GBS surface protein antigen attached to a GBS ferritin nanoparticle subunit would be considered a fusion protein of two heterologous polypeptide sequences.

“Operably linked” means connected so as to be operational, for example, in the configuration of recombinant polynucleotide sequences for protein expression. In certain embodiments, “operably linked” refers to the art-recognized positioning of nucleic acid components such that the intended function (e.g., expression) is achieved. A person with ordinary skill in the art will recognize that under certain circumstances, two or more components “operably linked” together are not necessarily adjacent to each other in the nucleic acid or amino acid sequence. A coding sequence that is “operably linked” to a control sequence (e.g., a promoter, enhancer, or IRES) is ligated in such a way that expression of the coding sequence is under the influence or control of the control sequence, but such a ligation is not limited to adjacent ligation.

By “adjacent”, it is meant “next to” or “side-by-side”. By “immediately adjacent”, it is meant adjacent to with no material structures in between (e.g., in the context of an amino acid sequence, two residues “immediately adjacent” to each other means there are atoms between the two residues sufficient to form the bonds necessary for a polypeptide sequence, but not a third amino acid residue).

By “c-terminally” or “c-terminal” to, it is meant toward the c-terminus. Therefore, by “c-terminally adjacent” it is meant “next to” and on the c-terminal side (i.e., on the right side if reading from left to right).

By “n-terminally” or “n-terminal” to, it is meant toward the n-terminus. Therefore, by “n-terminally adjacent” it is meant “next to” and on the n-terminal side (i.e., on the left side if reading from left to right).

As used herein, “mer” when referring to a protein nanoparticle, such as in means the number of subunit polypeptides that make up the NP. The subunit polypeptides do not have to be identical. Thus a 60-mer NP consists of sixty joined polypeptide subunits.

As used herein, a “recombinant” or “engineered” cell refers to a cell into which an exogenous DNA sequence, such as a cDNA sequence, has been introduced. A “host cell” is one that contains such an exogenous DNA sequence. “Recombinant” as used herein to describe a polynucleotide means a polynucleotide which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

A “subject” is a living multi-cellular vertebrate organism. In the context of this disclosure, the subject can be an experimental subject, such as a non-human mammal, e g , a mouse, a rat, or a non-human primate. Alternatively, the subject can be a human subject.

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “≈”) 200 pg.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is used herein to indicate a non-limiting example and is synonymous with the term “for example.”

It is further to be understood that all base sizes or amino acid sizes, and all molecular weight or molecular mass values, given for nucleic acid molecules or polypeptides are approximate and are provided for description. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated, a process comprising a step of mixing two or more components does not require any specific order of mixing. Thus components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not mean that the steps must be performed in that order (i.e., step 1 then step 2 then step 3, etc.). The word “then” may be used to specify the order of a method's steps.

The present invention is not limited to particular embodiments described herein. It is appreciated that certain features of the invention which are, for clarity, described in the context of separate embodiments, may also be provided in combination in a single embodiment. Conversely, various features of the invention which are, for brevity, described in the context of a single embodiment may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

Although methods and materials similar or equivalent to those described herein can be used in the practice or testing of this disclosure, suitable methods and materials are described below.

The entire disclosure of published references, patents, and published patent applications cited herein are incorporated herein by reference in their entirety.

### EXAMPLES

### Example 1

### Production of NPs

Three different nanoparticle scaffolds were compared: 1) GBS Ferritin NPs, which are made of 24 monomers, and have a diameter of 12-14 nm; 2) mI3 NPs, which are made of 60 copies of the trimeric building blocks i301 obtained engineering the 2-keto-3-deoxy-phosphogluconate (KDPG) aldolase from the hyperthermophilic bacterium Thermotoga maritima, and have a diameter of 25 nm, (Hsia et al., (2016)); and 3) QBeta NPs, which have essentially an icosahedral phage-like capsid structure with a diameter of about 35 nm, and are composed of 180 copies of coat protein linked in covalent pentamers and hexamers by disulfide bridges (Golmohammadi et al., (1996)).

QBeta NPs were produced by expression in E. coli cells, using polypeptides of SEQ ID NO:1 (PDB 5KIP):

| | |
|---|---|
| MAKLETVTLG NIGKDGKQTL VLNPRGVNPT NGVASLSQAG AVPALEKRVT VSVSQPSRNR - | 60 |
| KNYKVQVKIQ NPTACTANGS CDPSVTRQAY ADVTFSFTQY STDEERAFVR TELAALLASP - | 120 |
| LLIDAIDQLN PAY - | 133 |

DNA sequences encoding SEQ ID NO:1 were codon-optimized for expression in E. coli and cloned into pET21a vector. Transformed E. coli (Stellar™, Takara Bio) host cells were grown, and the plasmid DNA was extracted and sequenced in order to confirm the sequence identity. The plasmid was transformed into addition E. coli strains BL21DE3tlr and ClearColi™ (Lucigen), and the cells cultured. Material was purified using CAPTO Q column for ionic exchange chromatography with a NaCl salt gradient purification (from 0 to 1M NaCl). Fractions containing QBeta polypeptides were pooled and concentrated 6 times and further purified using size exclusion chromatography purification. Fractions were run on SDS page and those containing QBeta polypeptides were collected.

MI3 nanoparticles were produced using polypeptides of SEQ ID NO: 2 (see, e.g., Bruun et al., ACS Nano 12(9):8855-8866 (2018)):

| | |
|---|---|
| MKMEELFKKH KIVAVLRANS VEEAKKKALA VFLGGVHLIE ITFTVPDADT VIKELSFLKE - | 60 |
| MGAIIGAGTV TSVEQARKAV ESGAEFIVSP HLDEEISQFA KEKGVFYMPG VMTPTELVKA - | 120 |
| MKLGHTILKL FPGEVVGPQF VKAMKGPFPN VKFVPTGGVN LDNVCEWFKA GVLAVGVGSA - | 180 |
| LVKGTPVEVA EKAKAFVEKI RGCTE - | 205 |

The mI3 polypeptide was fused at its C-terminus to a peptide linker (GSGSGSGSGS—SEQ ID NO: 9) followed by a histidine tag to produce the mI3 polypeptide sequence of SEQ ID NO: 11:

MKMEELFKKHKIVAVLRANSVEEAKKKALAVFLGGVHLIEITFTVPDADT
VIKELSFLKEMGAIIGAGTVTSVEQARKAVESGAEFIVSPHLDEEISQFA
KEKGVFYMPGVMTPTELVKAMKLGHTILKLFPGEVVGPQFVKAMKGPFPN
VKFVPTGGVNLDNVCEWFKAGVLAVGVGSALVKGTPVEVAEKAKAFVEKI
RGCTEGSGSGSGSGSHHHHHH

DNA sequences encoding SEQ ID NO: 11 were codon-optimized for expression in E. coli and cloned into pET21 a vector. Transformed E. coli (Stellar™, Takara Bio) host cells were grown, and the plasmid DNA was extracted and sequenced in order to confirm the sequence identity. The plasmid containing the mI3-HIS was further transformed in E. coli strain BL21DE3t1r and cultured. Expressed polypeptides were purified with Affinity Chromatography and fractions containing mI3 were pooled and purified by Size Exclusion Chromatography.

GBS Ferritin NPs were produced using polypeptides of SEQ ID NO: 5, which contains a GBS ferritin protein from DK-PW-092 strain (amino acids 1-155 of SEQ ID NO: 5) followed by a peptide linker, GSSGH (SEQ ID NO: 10) and a C-terminus 6× histidine tag, to produce the GBS ferritin NP sequence of SEQ ID NO: 5:

| | |
|---|---|
| MKFEKTKEIL NQLVADLSQF SVVIHQTHWY MRGPEFLTLH PQMDEYMDQI NEQLDVVSER - | 60 |
| LITLDGSPFS TLREFAENTK IEDEIGNWDR TIPERMEKLV AGYRYLADLY AKGIEVSGEE - | 120 |
| GDDSTQDIFI ANKTDIEKNI WMLQAKLGKA PGIDAGSSGH HHHHH - | 165 |

### Example 2

### Production of GBS Capsular Oligosaccharides and Conjugation to NPs

A GBS CPS serotype II oligosaccharide (OS, molecular weight ˜10 kDa) was obtained by depolymerization through a three step de-N-acetylation/nitrosation/re-N-acetylation procedure (Michon et al (2006)).

Native serotype II PS was purified based on previously described procedure (Wessels et al. (1990)) and then partially N-deacylated as follows. The polysaccharide was dissolved in 0.5 M NaOH, heated at 70° C. for 2-4 h, and then chilled in an ice-water bath.

Glacial acetic acid was added to the sample to bring the pH to 4.5. The partially N-deacylated product was deaminated by the addition of 5% (wt/vol) NaNO₂ and stirred at 4° C. for 2 h. The material was purified by a G25 column eluting with water.

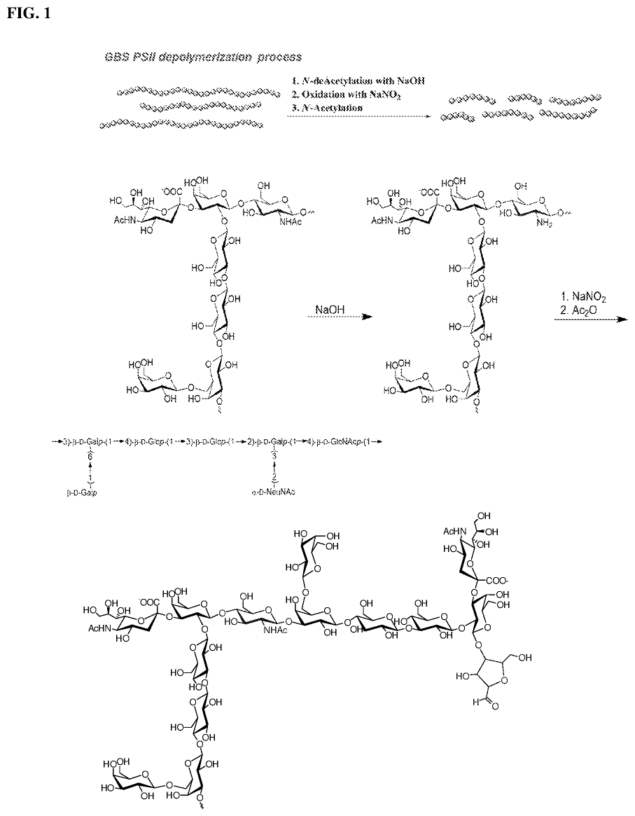

To reconstitute full N-acetylation of sialic acid residues, a 1:1 diluted solution of 4.15 μl/ml acetic anhydride in ethanol was added, and the reaction was incubated at room temperature for 2 h. The material was purified by a G25 column eluting with water. FIG. 1 depicts the depolymerization process including the structure of the obtained oligosaccharide.

The oligosaccharide fragments were separated by anionic exchange chromatography using FPLC system. Increasing the NaCl percentage of the elution buffer with a linear gradient, it was possible to isolate oligosaccharides with an average length between 6-14 repeating units.

The length of the oligosaccharides was determined by ¹H NMR analysis and SE-HPLC with pullulan standard curve. Total saccharide was quantified by HPAEC-PAD or Colorimetric assay (NeuNAc-based).

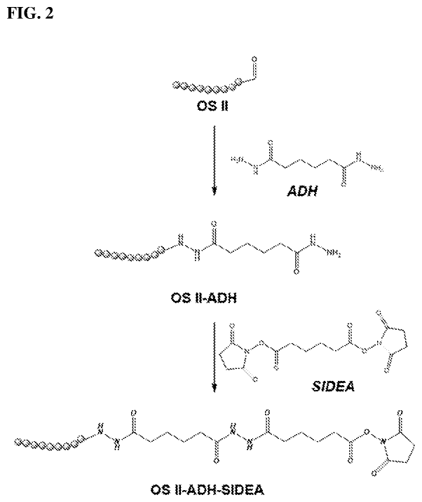

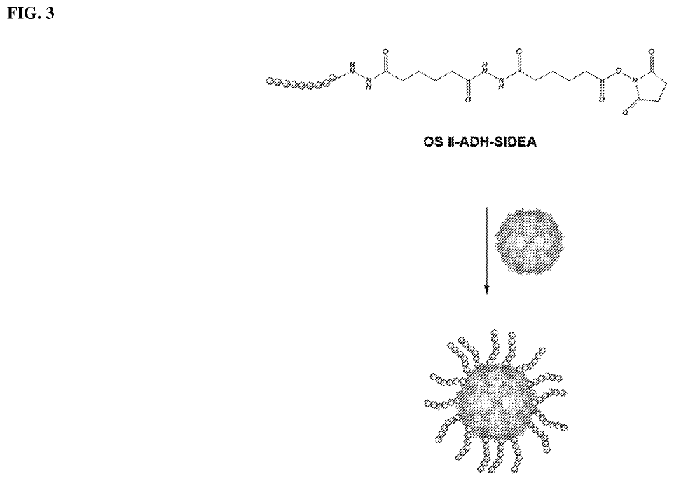

The GBS serotype II short oligosaccharides were modified with a hydrazine linker (ADH) by reductive amination followed by the addition of an active ester spacer (SIDEA), as shown in FIG. 2. These modified oligosaccharides were then conjugated to NPs, such as by incubating the derivatized oligosaccharides and highly concentrated nanoparticles (20-40mg/mL) at 15:1 or 30:1 (mol/mol), at room temperature, for approximately 16 hours in NaPi 10 mM pH 7.2. The final NPs conjugated to saccharides were purified by serial centrifugal filtration (100 kDa).

### Example 3

### Conjugation of GBS Capsular Polysaccharide to NPs

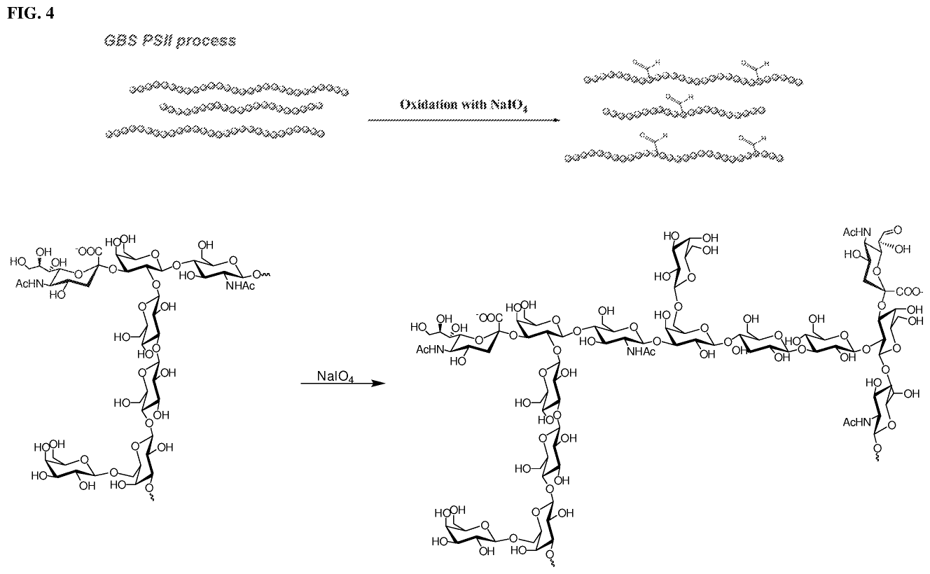

Polysaccharides of GBS CPS serotype II (molecular weight ˜400 kDa) were produced. Oxidation of GBS serotype II capsular polysaccharide was carried out using 5% of NaIO₄, as shown in FIG. 4. The oxidized polysaccharides were purified using a desalting column. Identity and structural conformity of the resulting polysaccharides were assessed by ¹H NMR. Total saccharide was quantified using HPAEC-PAD or Colorimetric assay (NeuNAc-based).

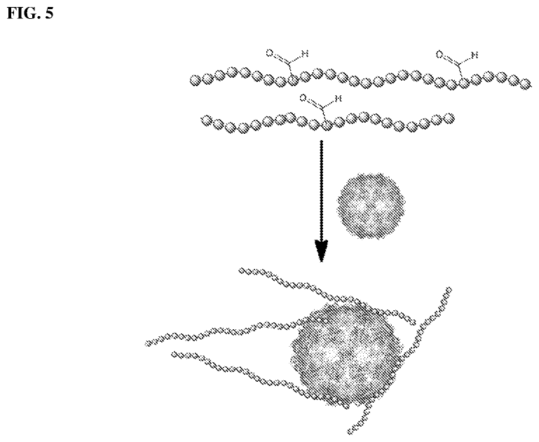

The oxidized polysaccharides were then conjugated to NPs (5-10mg/mL) at 37° C. for 72 hours by reductive amination in presence of NaBH₃CN, using a w/w ratio between saccharide and NP between 2:1 and 6:1 as illustrated in FIG. 5. The final NPs conjugated to saccharides were purified by ammonium sulfate precipitation followed by serial centrifugal filtration (100 kDa).

### Example 4

### GBS Saccharide NPs Conjugates Characterization

HPAEC-PAD and BCA were used to estimate saccharide (total and free) and protein content, respectively, of purified NPs conjugated to GBS saccharide, respectively, as reported in the Table 1 below.

TABLE 1

| | | | | Saccharide/ | |
|---|---|---|---|---|---|
| | | Saccharide | Protein | protein | Free saccharide |
| NP construct | Lot | (μg/mL) | (μg/mL) | (w/w) | % |
| OSII-GBS ferritin | FC12dic19 | 791 | 716 | 1.1 | <4.7 |
| PSII-GBS ferritin | FC21gen20 | 9518 | 1796 | 5.3 | 34.8 |
| OSII-ml3 | FC19lug19 | 385 | 602 | 0.6 | 11.5 |
| PSII-ml3 | FC21gen20 | 167 | 163 | 1.0 | 11.3 |
| OSII-QBeta | FC12set19 | 524 | 673 | 0.8 | 8.4 |
| PSII-QBeta | FC30set19 | 366 | 449 | 0.8 | <7.2 |

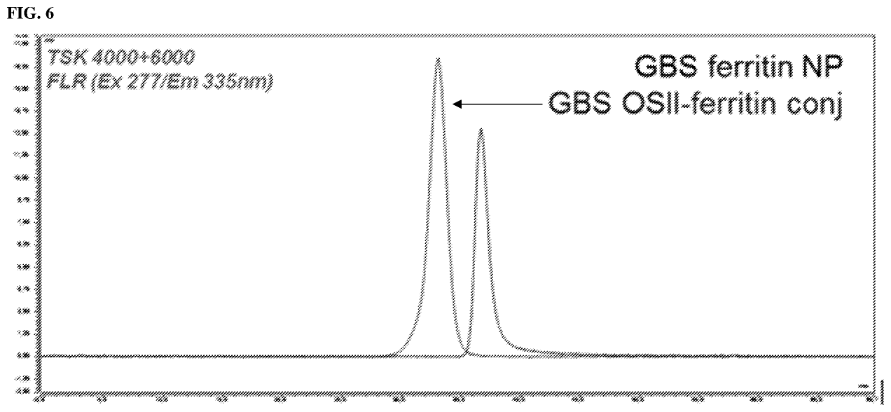

The OSII-ferritin NP conjugate (average MW 8 kDa) was produced using conjugate reaction conditions of OS:NP (mol/mol) 30:1, NP 37 mg/ml, at room temperature, for approximately 16 hours. FIG. 6 shows the SE-HPLC analysis of GBS OSII-ferritin NP conjugate, and the GBS ferritin NP (no conjugated saccharide).

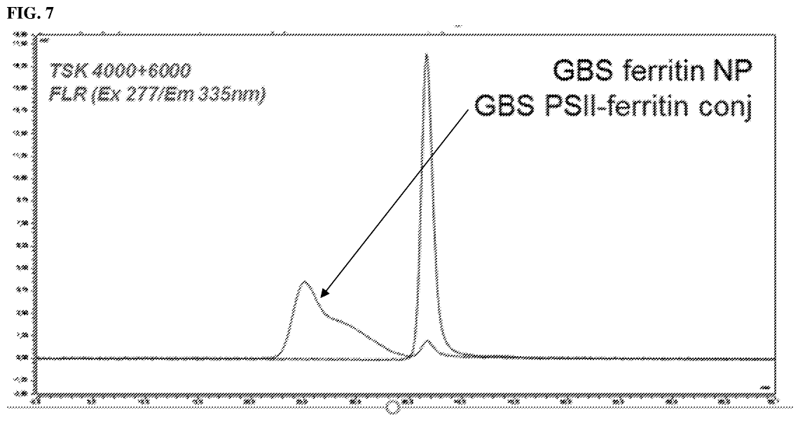

The PSII-ferritin NP conjugate was produced using conjugate reaction conditions of: PS:NP (w/w) 6:1, NP 6 mg/ml, at temperature 37° C., for approximately 72 hours. FIG. 7 shows the SE-HPLC analysis of GBS PSII-ferritin NP conjugate, and the GBS ferritin NP (no conjugated saccharide).

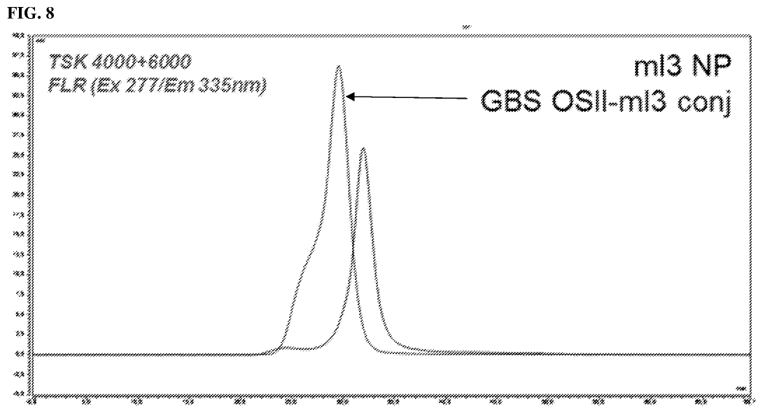

The OSII-mI3 NP conjugate (average MW 14 kDa) was produced using conjugate reaction conditions of OS:NP (mol/mol) 15:1, NP 23 mg/ml, at room temperature, for approximately 16 hours. FIG. 8 shows the SE-HPLC analysis of GBS OSII-mI3 NP conjugate, and the mI3 NP (no conjugated saccharide).

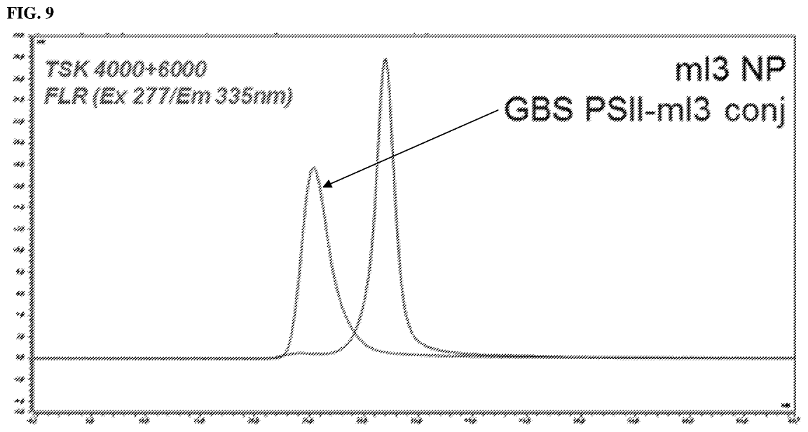

The PSII-mI3 NP conjugate was produced using conjugate reaction conditions of PS:NP (w/w) 2:1, NP 6 mg/ml, at temperature 37° C., for approximately 72 hours. FIG. 9 shows the SE-HPLC analysis of GBS PSII-mI3 NP conjugate, and the mI3 NP (no conjugated saccharide).

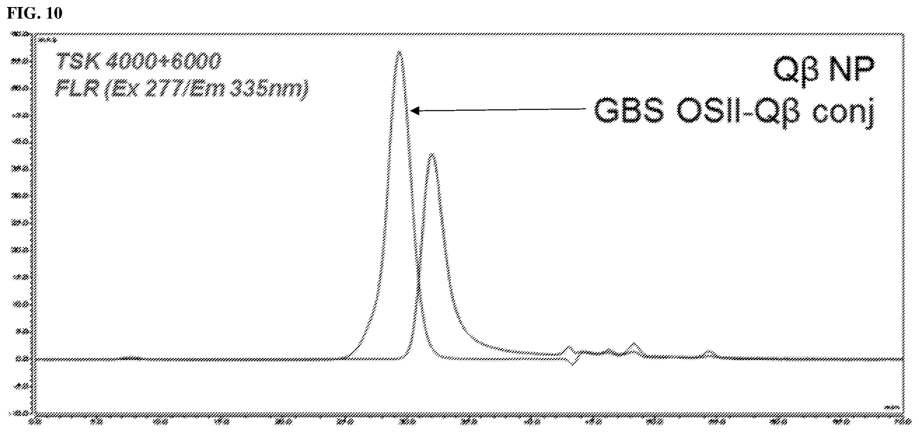

The OSII-QBeta NP conjugate (average MW 8 kDa) was produced using conjugate reaction conditions of OS:NP (mol/mol) 30:1, NP 23 mg/ml, at room temperature, for approximately 16 hours. FIG. 10 shows the SE-HPLC analysis of GBS OSII-QBeta NP conjugate, and the QBeta NP (no conjugated saccharide).

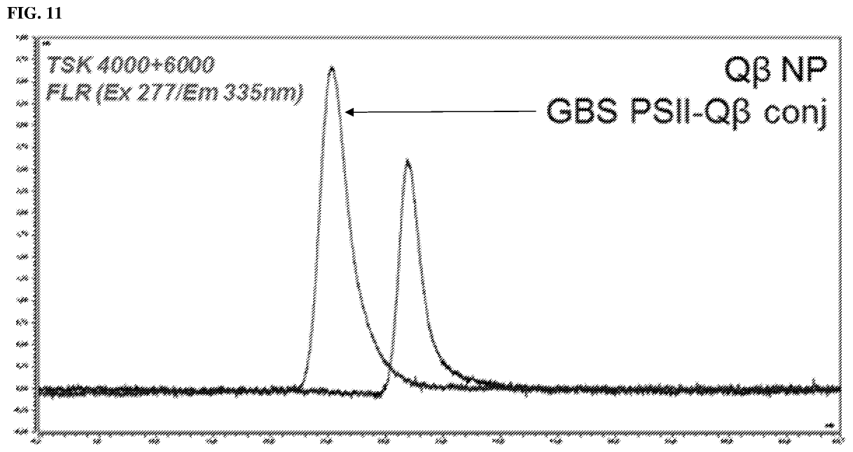

The PSII-QBeta NP conjugate was produced using conjugate reaction conditions of PS:NP (w/w) 4:1, NP 6 mg/ml, at temperature 37° C., for approximately 72 hours. FIG. 11 shows the SE-HPLC analysis of GBS PSII-QBeta NP conjugate, and the QBeta NP (no conjugated saccharide).

The NP conjugates were characterized in terms of purity by SDS-PAGE and SE-HPLC, in terms of size/structure by SE-HPLC, and in terms of identity by Western Blot experiments with a 11D3D2 PSII-specific murine monoclonal antibody. SE-HPLC was carried out using coupled TSK4000PW+TSK6000PW columns by Waters with fluorometric detection (excitation at 227 nm and emission at 335 nm). Running conditions were flow rate 0.5 mg/mL, run time 70 minutes, 100 mM NaPi, 100 mM Na2SO4, pH 7.2 as running buffer and injection volume20 μL. All samples were injected in a protein concentration of 0.3 mg/mL protein based.

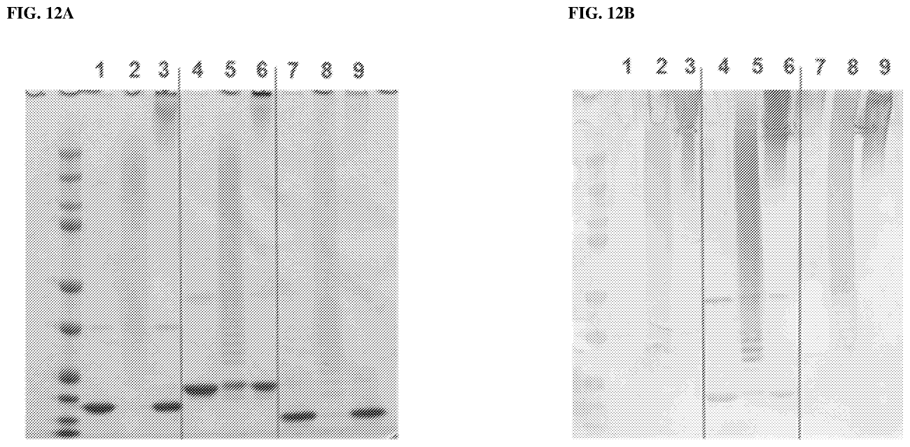

FIG. 12A shows results of SDS-PAGE (4-12% in MOPS), where lane 1 is GBS ferritin NP, lane 2 is OSII-GBS ferritin NP, lane 3 is PSII-GBS ferritin NP, lane 4 is mI3 NP, lane 5 is OSII-mI3 NP, lane 6 is PSII-mI3 NP, lane 7 is QBeta nanoparticle, lane 8 is OSII-QBeta NP, and land 9 is PSII-QBeta NP.

FIG. 12B provides Western Blot results, where lane 1 is GBS ferritin NP, lane 2 is OSII-GBS ferritin NP, lane 3 is PSII-GBS ferritin NP, lane 4 is mI3 NP, lane 5 is OSII-mI3 NP, lane 6 is PSII-mI3 NP, lane 7 is QBeta nanoparticle, lane 8 is OSII-QBeta NP, and land 9 is PSII-QBeta NP.

NPs conjugated to GBS saccharides were also characterized by transmission electron microscopy (TEM) analysis, using negative stain (NS) and immunogold staining. For analysis by negative staining, NPs conjugated to GBS oligosaccharides and polysaccharides were loaded onto copper 300-square mesh grids of carbon/formvar (Agar Scientific) rendered hydrophilic by glow discharge (Quorum Q150). The excess solution was blotted off using Whatman filter Paper No.1 and then the grids were negatively stained with NanoW. Micrographs were acquired using a Tecnai G2 Spirit Transmission Electron Microscope at 87000× magnification equipped with a CCD 2k×2k camera.

For analysis by immunogold staining, purified nanoparticle conjugates with a final concentration of 20 ng/μL were adsorbed to 300-mesh nickel grids (Agar Scientific), blocked in Phosphate Buffered Saline (PBS) with 0.5% bovine serum albumin (BSA) and incubated with 11D3D2 PSII-specific murine monoclonal antibody (diluted 1:1000 or 1:2000 in PBS with 0.5% BSA) for 1 hour. Grids were washed several times and incubated with 10-nm gold-labeled anti-mouse secondary antibody (diluted 1:40 in PBS with 0.5% BSA) for 1 hour. After several washes with distilled water the grids were negatively stained with NanoW and observed using a TEM FEI Tecnai G2 Spirit microscope operating at 100 kV and equipped with an 2K×2K CCD Emsis Veleta camera (Emsis, Germany). Images were acquired and processed using iTem (OSIS, Olympus, Shinjuku, Tokyo, Japan) software.

Negative stain TEM images of ferritin NPs conjugated to GBS OSII showed a typical octahedral symmetry with a diameter around 12 nm. Immunogold stain TEM images of ferritin NPs conjugated to GBS OSII showed nanoparticles lightly labelled by the murine Mab11D3D2.

Negative stain TEM images of ferritin NPs conjugated to GBS PSII showed a typical octahedral symmetry with a diameter around 12 nm. Immunogold stain TEM images of ferritin NPs conjugated to GBS PSII showed nanoparticles heavily labelled by the murine Mab11D3D2. The presence of elongated PSII gold labelled appendages on the NPs were observed.

Negative stain TEM images of mI3 nanoparticles conjugated to GBS OSII showed a typical dodecahedral symmetry with a diameter around 18 nm. Immunogold stain TEM images showed GBS OSII on the surface of the dodecahedral MI3 nanoparticles when labelled by gold-labelled secondary antibodies binding murine Mab11D3D2 primary antibody diluted at 1:1000 and at 1:2000.

Negative stain TEM images of mI3 nanoparticles conjugated to GBS PSII showed a typical dodecahedral symmetry with a diameter around 18 nm, with a few thin detached appendages corresponding to GBS PSII visible in the background. Immunogold stain TEM images of MI3 nanoparticles conjugated to GBS PSII were obtained.

Negative stain TEM images of QBeta nanoparticles conjugated to GBS OSII showed typical icosahedral symmetry with a diameter around 33 nm. Immunogold stain TEM images showed GBS OSII on the surface of the icosahedral QBeta nanoparticles are labelled by 10 nm gold-labelled secondary antibodies binding murine Mab11D3D2 primary antibody.

Negative stain TEM images of QBeta nanoparticles conjugated to GBS PSII showed typical icosahedral symmetry with a diameter around 33 nm, with thin, elongated appendages (up to 20 nm in length) corresponding to PSII attached to the QBeta surface. Some detached appendages were visible in the background. Immunogold stain TEM images showed the GBS PSII on the surface of the icosahedral QBeta nanoparticles labelled by 10 nm gold-labelled secondary antibodies binding murine Mab11D3D2 primary antibody.

Thus, GBS ferritin, mI3 and QBeta nanoparticles visualized by Negative Stain Transmission Electron Microscopy (NS-TEM) appear as highly symmetrical structures. Negative stain electron microscopy of octahedral ferritin, dodecahedral MI3 and icosahedral QBeta revealed increased diameters for all conjugated nanoparticles compared with their unconjugated counterparts. The OSII conjugated nanoparticles showed thin and short appendages with an average length of 8-15 nm corresponding approximately to about 6 to 10 GBS type II repeating units. The OSII appear to be distributed on the scaffold following the different symmetry of the NPs. In the PSII-conjugated nanoparticles, the PSII could be found either detached and present in the background, or as decorating the nanoparticles as thin and long appendages with an average length of 400 nm corresponding to a GBS type II polysaccharide composed of about 300 repeating units. The distribution of PSII on the NPs resembled that observed for OSII.

### Example 5

### In Vivo Immunization

An in vivo mouse immunization study (Study 1) was conducted, using different forms of GBS serotype II antigen, either conjugated to CRM197 carrier protein, or conjugated to one of two different nanoparticles, mI3, or QBeta Immunizations and blood draws were carried out according to the schedule set forth in Table 2.

TABLE 2

| Day | Action |
|---|---|
| 0 | Blood Draw 1 (pre-immunization) |
| 1 | Immunization 1 |
| 21 | Blood Draw 2 (post first immunization) |
| 22 | Immunization 2 |
| 36 | Blood Draw Final (post second immunization) |

In Study 1, twelve groups of ten female mice each (CD1 strain, Charles River) were studied. Each mouse was immunized twice intraperitoneally with the formulations as shown in Table 3. Immunizations were carried out at Day 1 and Day 22. Blood was drawn from each mouse on Day 0 (pre-immunization), Day 21, and Day 36, as described in Table 2.

Table 4 shows the Geometric Mean IgG Titers in sera as measured by Luminex for Study 1, along with Opsonophagocytic Killing Titers obtained with pooled sera from each group.

TABLE 3

| Group | Antigen | Antigen dose | Adjuvant |
|---|---|---|---|
| 1 | None | | Alum 2 mg/mL |
| 2 | PSII-CRM | 0.5 μg GBSII | Alum 2 mg/mL |
| 3 | mi3 | 0.8 μg protein | none |
| 4 | mi3 | 0.8 μg protein | Alum 2 mg/mL |
| 5 | QBeta | 0.6 μg protein | none |
| 6 | QBeta | 0.6 μg protein | Alum 2 mg/mL |
| 7 | OSII-mi3 | 0.5 μg GBSII | none |
| 8 | OSII-mi3 | 0.5 μg GBSII | Alum 2 mg/mL |
| 9 | OSII-QBeta | 0.5 μg GBSII | none |
| 10 | OSII-QBeta | 0.5 μg GBSII | Alum 2 mg/mL |
| 11 | PSII-QBeta | 0.5 μg GBSII | none |
| 12 | PSII-QBeta | 0.5 μg GBSII | Alum 2 mg/mL |

TABLE 4
Study 1 IgG Titers (pooled sera)

| | | | Adjuvant | Luminex IgG GMT Titer in Sera | | | | |
|---|---|---|---|---|---|---|---|---|
| | | | (Alum 2 | [RLU/ml] | | | OPKA titers | |
| | Mice | | mg/mL or | PI_ | Post1_ | Post2_ | Post1_ | Post2_ |
| Group | CD1 | Antigen | none) | Day0 | day21 | day36 | day21 | day36 |
| 1 | 1-10 | None | Alum | <LLOQ | 26.2 | 32.7 | 32 | <30 |
| 2 | 11-20 | PSII-CRM | Alum | <LLOQ | 565.4 | 10490.9 | 429 | 2073 |
| 3 | 21-30 | mi3 | none | <LLOQ | 10.2 | 20.4 | <30 | <30 |
| 4 | 31-40 | mi3 | Alum | <LLOQ | 10.2 | 10.2 | <30 | <30 |
| 5 | 41-50 | QBeta | none | <LLOQ | 10.2 | 10.2 | <30 | <30 |
| 6 | 51-60 | QBeta | Alum | <LLOQ | 23.0 | 47.3 | <30 | <30 |
| 7 | 61-70 | OSII-mi3 | none | <LLOQ | 190.5 | 1832.4 | 315 | 663 |
| 8 | 71-80 | OSII-mi3 | Alum | <LLOQ | 77.7 | 4338.7 | 193 | 603 |
| 9 | 81-90 | OSII- | none | <LLOQ | 509.8 | 6217.6 | 126 | 582 |
| | | QBeta | | | | | | |
| 10 | 91- | OSII- | Alum | <LLOQ | 4777.0 | 74947.6 | 2252 | 15541 |
| | 100 | QBeta | | | | | | |
| 11 | 101- | PSII-QBeta | none | <LLOQ | 6543.5 | 50735.1 | 431 | 2413 |
| | 110 | | | | | | | |
| 12 | 111- | PSII-QBeta | Alum | <LLOQ | 1468.4 | 17398.8 | 1339 | 4628 |
| | 120 | | | | | | | |

Luminex Lower Limit of Quantification (LLOQ) = 20.4 Relative Luminex Units/ml; <LLOQ = 10.2

Serum antibody titers in serum were measured by a Luminex assay using streptavidin-derivatized magnetic microspheres (Radix Biosolutions, USA) coupled with biotinylated type II native polysaccharide (Buffi et al., (2019)). Following equilibration at RT, 1.25 million microspheres were transferred to LoBind tubes (Eppendorf) and placed into a magnetic separator for 2 min in the dark. Microspheres were washed with PBS containing 0.05% TWEEN™ 20 (Calbiochem) and biotin-PSII was added to the microspheres at a final concentration of 1 μg/ml in PBS, 0.05% TWEEN™ 20, 0.5% BSA (Sigma-Aldrich). The biotin-PSII—microspheres were incubated for 60 minutes at Room Temperature (RT) in the dark and washed twice with PBS, 0.05% TWEEN™ 20. Coupled microspheres were suspended in 500 μl of PBS, 0.05% TWEEN™ 20, 0.5% BSA and stored at 4° C.

Eight 3-fold serial dilutions of a standard hyperimmune serum or test samples were prepared in PBS, pH 7.2, 0.05% TWEEN™ 20, 0.5% BSA. Each serum dilution (50 μl) was mixed with an equal volume of conjugated microspheres (3,000 microspheres/region/well) in a 96-well Greiner plate (Millipore Corporation) and incubated for 60 min at RT in the dark. After incubation, the microspheres were washed three times with 200 μl PBS. Each well was loaded with 50 μl of 2.5 μg/ml anti-mouse IgG secondary antibody (Jackson Immunoresearch), in PBS, pH 7.2, 0.05% TWEEN™ 20, 0.5% BSA and incubated for 60 min with continuous shaking. After washing, microspheres were suspended in 100 μl PBS and shaken before the analysis with a Luminex 200 instrument. Data were acquired in real time by Bioplex Manager™ Software (BioRad).

The functional activity of the sera was determined by Opsonophagocytic Killing Assay (OPKA) as previously described (Chatzikleanthous (2020)). HL60 cells were grown in RPMI 1640 with 10% fetal calf serum, incubated at 37° C., 5% CO2. HL-60 cells were differentiated to neutrophils with 0.78% dimethylformamide (DMF) and after 4-5 days were used as source of phagocytes. The assay was conducted in 96-well microtiter plate, in a total volume of 125 μL/well. Each reaction contained heat inactivated test serum (12.5 μL), GBS II strain 5401 (6×104 colony forming units [CFU]), differentiated HL-60 cells (2×10⁶ cells) and 10% baby rabbit complement (Cederlane) in Hank's balanced salt solution red (Gibco). For each serum sample, six serial dilutions were tested. Negative controls lacked effector cells, or contained either negative sera or heat inactivated complement. After reaction assembly, plates were incubated at 37° C. for 1 hour under shaking. Before (T0) and after (T60) incubation, the mixtures were diluted in sterile water and plated in Trypticase Soy Agar plates with 5% sheep blood (Becton Dickinson). Each plate was then incubated overnight at 37° C. with 5% of CO₂; CFUs were counted the next day. OPKA titre was expressed as the reciprocal serum dilution leading to 50% killing of bacteria and the % of killing is calculated as follows

$$\% killing = \frac{{T}_{0} - {T}_{60}}{{T}_{0}}$$

where T₀ is the mean of the CFU counted at T₀ and T₆₀ is the average of the CFU counted at T₆₀ for the two replicates of each serum dilution.

It was noted that Post-1 IgG Luminex titers in groups 10 and 12 receiving Qbeta-PS or -OS conjugates formulated in Alum were significantly higher than post-1 Titers from group 2 receiving PSII-CRM in Alum. After one vaccine dose, OPK titers in pooled sera from animals receiving one dose of Qbeta conjugates (groups 10 and 12) were above 3-fold than those receiving 1 dose of PSII-CRM (group 2) and non-inferior (comparable) to the same group receiving two vaccine doses.

### Example 6

### In Vivo Immunization

Six groups of CD1 mice were immunized via either intraperitoneal (IP) or intramuscular (IM) route of administration, according to the schedule shown in Table 5, using the formulations shown in Table 6 (Study 2). Groups 1 and 2 (five mice each) received only Aluminum hydroxide adjuvant, without any GBS antigen. Table 7 shows serum antibody IgG Titers (pooled sera), measured by Luminex assay as described herein.

TABLE 5
Study 2

| Day | Action |
|---|---|
| 0 | Blood Draw 1 (pre-immunization) |
| 1 | Immunization 1 |
| 21 | Blood Draw 2 (post first immunization) |
| 22 | Immunization 2 |
| 36 | Blood Draw Final (post second immunization) |

TABLE 6
Study 2

| Group | GBS Antigen | Antigen (GBSII) dose | Adjuvant | Route |
|---|---|---|---|---|
| 1 | None | None | Alum 2 mg/mL | IP |
| 2 | None | none | Alum 2 mg/mL | IM |
| 3 | PS-CRM | 0.5 μg (saccharide) | Alum 2 mg/mL | IP |
| 4 | PS-CRM | 0.5 μg (saccharide) | Alum 2 mg/mL | IM |
| 5 | OS-QBeta | 0.5 μg (saccharide) | Alum 2 mg/mL | IP |
| 6 | OS-QBeta | 0.5 μg (saccharide) | Alum 2 mg/mL | IM |

TABLE 7
IgG Titers (pooled sera)-Study 2

| | | | | Luminex IgG Titer (pooled sera) | | |
|---|---|---|---|---|---|---|
| | Mice | | | P1_ | P1_ | P2_ |
| Group | CD1 | Antigen | Route | Day 1 | Day 21 | Day 36 |
| 1 | 1-5 | [Alum only] | IP 200 μl | <LLOQ | <LLOQ | <LLOQ |
| 2 | 6-10 | [Alum only] | IM 50 μl | <LLOQ | LLOQ | LLOQ |
| 3 | 11-20 | PSII-CRM | IP 200 μl | <LLOQ | 565.9 | 15667.2 |
| 4 | 21-30 | PSII-CRM | IM 50 μl | <LLOQ | 374.1 | 9606.9 |
| 5 | 31-40 | OSII-QBeta | IP 200 μl | <LLOQ | 24279.3 | 389571.0 |
| 6 | 41-50 | OSII-QBeta | IM 50 μl | <LLOQ | 26537.4 | 334436.5 |

Luminex LLOQ = 20.4 RLU/ml; <LLOQ = 10.2

It was noted that post-1 IgG Luminex titers in group 5 receiving OS-II conjugate via IP were significantly higher than post-1 Titers from group 3 receiving PSII-CRM via the same route and non-inferior (comparable) to group 3 post-2 doses. Similarly, post-1 IgG Luminex titers in group 6 receiving OS-II conjugate via IM were significantly higher to post-1 titers from group 4 receiving PSII-CRM via the same route and non-inferior (comparable) to group 4 post-2 doses.

### Example 7

### In Vivo Immunization

Two different in vivo mouse immunization studies were conducted (Study 3 and 4), using different forms of GBS serotype II antigen, either conjugated to CRM197 carrier protein, or conjugated to one of three different nanoparticles (GBS Ferritin, mI3, or QBeta) Immunizations and blood draws were carried out according to the schedule set forth in Table 8.

TABLE 8
Study 3 and Study 4

| Day | Action |
|---|---|
| 0 | Blood Draw 1 (pre-immunization) |
| 1 | Immunization 1 |
| 21 | Blood Draw 2 (post first immunization) |
| 22 | Immunization 2 |
| 36 | Blood Draw Final (post second immunization) |

In Study 3, nine groups of ten mice each (CD1 strain, Charles River) were studied. Each mouse was immunized twice intramuscularly with the formulations as shown in Table 9. Immunizations were carried out at Day 1 and Day 22. Blood was drawn from each mouse on Day 0 (pre-immunization), Day 21, and Day 36, as described in Table 8.

Table 10 shows serum antibody IgG Titers (pooled sera), measured by Luminex assay as described herein.

TABLE 9
Study 3

| | | Antigen | |
|---|---|---|---|
| Group | Antigen | (GBSII) dose | Adjuvant |
| 1 | PSII-CRM | 0.5 μg | none |
| 2 | PSII-CRM | 0.5 μg | Alum 2 mg/mL |
| 3 | OSII-GBS ferritin | 0.5 μg | none |
| 4 | PSII-GBS ferritin | 0.5 μg | none |
| 5 | OSII-mI3 | 0.5 μg | none |
| 6 | PSII-mI3 | 0.5 μg | none |
| 7 | OSII-QBeta | 0.5 μg | none |
| 8 | PSII-QBeta | 0.5 μg | none |
| 9 | PSII-CRM | 0.5 μg | Alum 2 mg/mL |

TABLE 10
Study 3 IgG Titers (pooled sera)

| | Mice | | | Luminex IgG Titer Pooled Sera RLU/ml | | |
|---|---|---|---|---|---|---|
| Group | CD1 | Antigen | Adjuvant | PI_Day0 | Post1_Day21 | Post2_Day36 |
| 1 | 1-10 | PSII-CRM GBD- | None | <LLOQ | 319.8 | 9000.7 |
| | | CRM004 | | | | |
| 2 | 11-20 | PSII-CRM GBD- | Alum | <LLOQ | 185.4 | 4382.9 |
| | | CRM004 | 2 mg/mL | | | |
| 3 | 21-30 | OSII-GBS ferritin | None | <LLOQ | 147.7 | 6801.2 |
| 4 | 31-40 | PSII-GBS ferritin | None | <LLOQ | 45.1 | 102.6 |
| 5 | 41-50 | OSII-ml3 | None | <LLOQ | 43.4 | 1379.6 |
| 6 | 51-60 | PSII-ml3 | None | <LLOQ | 347.8 | 5402.1 |
| 7 | 61-70 | OSII-QBeta | None | <LLOQ | 3206.6 | 69392.8 |
| 8 | 71-80 | PSII-QBeta | None | <LLOQ | 5682.4 | 59662.0 |
| 9 | 81-90 | PSII-CRM lot EB | Alum | <LLOQ | 314.2 | 5265.3 |
| | | | 2 mg/mL | | | |

Luminex Lower Limit of Quantification (LLOQ) = 20.4 Relative Luminex Units/ml; <LLOQ = 10.2

It was noted that post-1 IgG Luminex titers in groups 7 and 8 receiving OS-Qbeta and PS-Qbeta without Alum conjugates respectively were significantly higher than post-1 Titers from group 1 receiving PSII-CRM without Alum via the same route and non-inferior (comparable) to group 1 post-2 doses.

In Study 4, eight groups of ten mice each (CD1 strain, Charles River) were studied. Each mouse was immunized twice intramuscularly with the formulations as shown in Table 11. Immunizations were carried out at Day 1 and Day 22. Blood was drawn from each mouse on Day 0 (pre-immunization), Day 21, and Day 36, as described in Table 8.

Table 12a shows serum antibody IgG Titers (pooled sera), measured by Luminex assay as described herein.

TABLE 11
Study 4

| | | Antigen | |
|---|---|---|---|
| Group | Antigen | (GBSII) dose | Adjuvant |
| 1 | PSII-CRM | 0.5 μg | Alum 2 mg/mL |
| 2 | OSII-GBS ferritin | 0.5 μg | Alum 2 mg/mL |
| 3 | PSII-GBS ferritin | 0.5 μg | Alum 2 mg/mL |
| 4 | OSII-mI3 | 0.5 μg | Alum 2 mg/mL |
| 5 | PSII-mI3 | 0.5 μg | Alum 2 mg/mL |
| 6 | OSII-QBeta | 0.5 μg | Alum 2 mg/mL |
| 7 | PSII-QBeta | 0.5 μg | Alum 2 mg/mL |
| 8 | PSII-CRM | 0.5 ng | Alum 2 mg/mL |

TABLE 12a
Study 4 IgG Titers (pooled sera)

| | | | | Luminex IgG Titer Pooled Sera | | |
|---|---|---|---|---|---|---|
| | Mice | | | RLU/ml | | |
| Group | CD1 | Antigen | Adjuvant | PI_Day0 | Post1_day21 | Post2_day36 |
| 1 | 1-10 | PSII-CRM GBD- | Alum 2 mg/mL | <LLOQ | 587.1 | 8774.1 |
| | | CRM004 | | | | |
| 2 | 11-20 | OSII-GBS ferritin | Alum 2 mg/mL | <LLOQ | 299.3 | 18985.0 |
| 3 | 21-30 | PSII-GBS ferritin | Alum 2 mg/mL | <LLOQ | 1294.6 | 8341.4 |
| 4 | 31-40 | OSII-ml3 | Alum 2 mg/mL | <LLOQ | 398.0 | 23556.6 |
| 5 | 41-50 | PSII-ml3 | Alum 2 mg/mL | <LLOQ | 542.2 | 4087.8 |
| 6 | 51-60 | OSII-QBeta | Alum 2 mg/mL | <LLOQ | 9531.5 | 151102.3 |
| 7 | 61-70 | PSII-QBeta | Alum 2 mg/mL | <LLOQ | 4160.0 | 15720.7 |
| 8 | 71-80 | PSII-CRM lot EB | Alum 2 mg/mL | <LLOQ | 470.9 | 7732.7 |

Luminex LLOQ = 20.4 RLU/ml; <LLOQ = 10.2

It was noted that post-1 IgG Luminex titers in groups 6 and 7 receiving OS-Qbeta and PS-Qbeta Alum conjugates respectively were significantly higher than post-1 Titers from group 1 receiving PSII-CRM with Alum via the same route and non-inferior (comparable) to group 1 post-2 doses.

Comparing IgG titers from Studies 3 and 4:

TABLE 12b
comparison of Studies 3 and 4

| Exp/ | | | Post 1 | Post 1 | Post 2 |
|---|---|---|---|---|---|
| Group | Antigen | Adjuvant | Day 0 | Day 21 | Day 36 |
| 3 | PSII-CRM GBD- | None | <LLOQ | 319.8 | 9000.7 |
| 3 | CRM004 | Alum 2 mg/mL | <LLOQ | 185.4 | 4382.9 |
| 4 | | Alum 2 mg/mL | <LLOQ | 587.1 | 8774.1 |
| 3 | PSII-CRM lot EB | Alum 2 mg/mL | <LLOQ | 314.2 | 5265.3 |
| 4 | | Alum 2 mg/mL | <LLOQ | 470.9 | 7732.7 |
| 3 | OSII-GBS ferritin | none | <LLOQ | 147.7 | 6801.2 |
| 4 | | Alum 2 mg/mL | <LLOQ | 299.3 | 18985.0 |
| 3 | PSII-GBS ferritin | none | <LLOQ | 45.1 | 102.6 |
| 4 | | Alum 2 mg/mL | <LLOQ | 1294.6 | 8341.4 |
| 3 | OSII-mI3 | none | <LLOQ | 43.4 | 1379.6 |
| 4 | | Alum 2 mg/mL | <LLOQ | 398.0 | 23556.6 |
| 3 | PSII-MI3 | none | <LLOQ | 347.8 | 5402.1 |
| 4 | | Alum 2 mg/mL | <LLOQ | 542.2 | 4087.8 |
| 3 | OSII-QBeta | none | <LLOQ | 3206.6 | 69392.8 |
| 4 | | Alum 2 mg/mL | <LLOQ | 9531.5 | 151102.3 |
| 3 | PSII-QBeta | none | <LLOQ | 5682.4 | 59662.0 |
| 4 | | Alum 2 mg/mL | <LLOQ | 4160.0 | 15720.7 |

### Example 8

### In Vivo Immunization

Ten groups of CD1 mice (ten mice per group) were immunized via intramuscular administration according to the schedule shown in Table 13, using the formulations shown in Table 14. Table 15 shows serum antibody IgG Titers (pooled sera), measured by Luminex assay as described herein.

TABLE 13
in vivo immunization

| Day | Action |
|---|---|
| 0 | Blood Draw 1 (pre-immunization) |
| 1 | Immunization 1 |
| 21 | Blood Draw 2 (post first immunization) |
| 22 | Immunization 2 |
| 36 | Blood Draw Final (post second immunization) |

TABLE 14
in vivo immunization

| Group | GBS Antigen | Antigen Dose | Adjuvant |
|---|---|---|---|
| 1 | PSII-CRM | 0.01 μg | Alum 2 mg/mL |
| 2 | PSII-CRM | 0.1 μg | Alum 2 mg/mL |
| 3 | PSII-CRM | 0.5 μg | Alum 2 mg/mL |
| 4 | PSII-CRM | 1.0 μg | Alum 2 mg/mL |
| 5 | OSII-QBeta | 0.01 μg | Alum 2 mg/mL |
| 6 | OSII-QBeta | 0.1 μg | Alum 2 mg/mL |
| 7 | OSII-QBeta | 1.0 μg | Alum 2 mg/mL |
| 8 | PSII-QBeta | 0.01 μg | Alum 2 mg/mL |
| 9 | PSII-QBeta | 0.1 μg | Alum 2 mg/mL |
| 10 | PSII-QBeta | 1.0 μg | Alum 2 mg/mL |

CRM = CRM197

TABLE 15
IgG Titers (pooled sera)-in vivo immunization

| | Mice | | GBSII antigen | Luminex IgG Titer (pooled sera) | | |
|---|---|---|---|---|---|---|
| Group | CD1 | GBS Antigen | dose | P1_Day 1 | P1_Day 21 | P2_Day 36 |
| 1 | 1-10 | PSII-CRM | 0.01 μg | <LLOQ | 161.3 | 984.13 |
| 2 | 11-20 | PSII-CRM | 0.1 μg | <LLOQ | 374.9 | 15324.19 |
| 3 | 21-30 | PSII-CRM | 0.5 μg | <LLOQ | 406.0 | 13143.61 |
| 4 | 31-40 | PSII-CRM | 1.0 μg | <LLOQ | 103.8 | 3690.86 |
| 5 | 41-50 | OSII-QBeta | 0.01 μg | <LLOQ | 976.2 | 21097.64 |
| 6 | 51-60 | OSII-QBeta | 0.1 μg | <LLOQ | 5198.4 | 87351.08 |
| 7 | 61-70 | OSII-QBeta | 1.0 μg | <LLOQ | 13924.9 | 230182.80 |
| 8 | 71-80 | PSII-QBeta | 0.01 μg | <LLOQ | 1232.1 | 7593.40 |
| 9 | 81-90 | PSII-QBeta | 0.1 μg | <LLOQ | 1007.6 | 5268.70 |
| 10 | 91-100 | PSII-QBeta | 1.0 μg | <LLOQ | 2914.8 | 37367.20 |

Luminex LLOQ = 20.4 RLU/ml; <LLOQ = 10.2

This dose ranging experiment compared administration of 0.1, 0.5, and 1.0 μg GBS saccharide antigen, given in a two-dose schedule. The antigens were provided as either conjugates of polysaccharide and CRM197 (PSII-CRM), QBeta NPs displaying oligosaccharides (OSII-QBeta), or QBeta NPs displaying polysaccharides (PSII-QBeta). All administrations were adjuvanted with alum.

In mice receiving PSII-CRM, after the first administration, anti-CPSII IgG titers were lower in mice receiving the highest (1.0 μg) dose, compared to the smaller doses of PSII-CRM. After the second administration, anti-CPSII IgG titers decreased as the dose increased from 0.1 μg to 1.0 μg. In contrast, in mice receiving OSII-QBeta, anti-CPSII IgG titers increased in a dose-dependent manner In mice receiving PSII-QBeta, anti-CPSII IgG titers were highest in the group receiving the highest (1.0 μg) dose after both the first and second administration.

### Example 10

### GBS PSIa-OBeta Conjugates

Conjugation of GBS Capsular Polysaccharide to QBeta VLPs

Polysaccharides of GBS CPS serotype Ia (molecular weight ˜100 kDa) were produced. Oxidation of GBS serotype Ia capsular polysaccharide was carried out using 20% of NaIO4. The oxidized polysaccharides were purified using a desalting column. Identity and structural conformity of the resulting polysaccharides were assessed by 1H NMR. Total saccharide was quantified using HPAEC-PAD or Colorimetric assay (NeuNAc based).

The oxidized polysaccharides were then conjugated to QBeta VLPs (5-10mg/mL) at 37° C. for 72 hours by reductive amination in presence of NaBH3CN, using a w/w ratio between saccharide and NP between 2:1 and 6:1. The final NPs conjugated to saccharides were purified by tangential flow filtration using a Hydrosart membrane 100 kDa cutoff.

GBS Saccharide VLP Conjugates Characterization

HPAEC-PAD and BCA were used to estimate saccharide (total and free) and protein content, respectively, of purified QBeta VLPs conjugated to GBS saccharide, respectively, as reported in the Table 16 below.

TABLE 16

| | | Sac- | | Saccharide/ | Free |
|---|---|---|---|---|---|
| NP | | charide | Protein | protein | saccharide |
| construct | Lot | (μg/mL) | (μg/mL) | (w/w) | % |
| PSIa-QBeta | FC11ago20 | 201.4 | 464.0 | 0.43 | 12.1 |

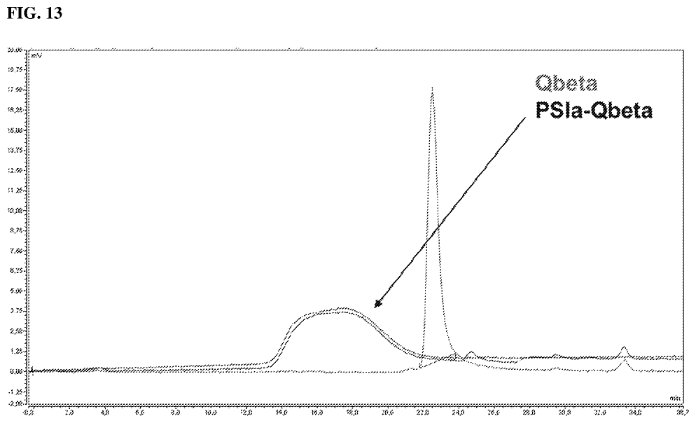

The PSIa-QBeta NP conjugate was produced using conjugate reaction conditions of PS:NP (w/w) 2:1, NP 6 mg/ml, at temperature 37° C., for approximately 72 hours. FIG. 13 shows the SE-HPLC analysis of GBS PSIa-QBeta NP conjugate, and the QBeta NP (no conjugated saccharide).

SE-HPLC was carried out using SRT-C 2000 column with fluorometric detection (excitation at 227 nm and emission at 335 nm). Running conditions were flow rate 0.5 mg/mL, run time 40 minutes, 100 mM NaPi, 100 mM Na2SO4, pH 7.2 as running buffer and injection volume20 μL. All samples were injected in a protein concentration of 0.3 mg/mL protein based.

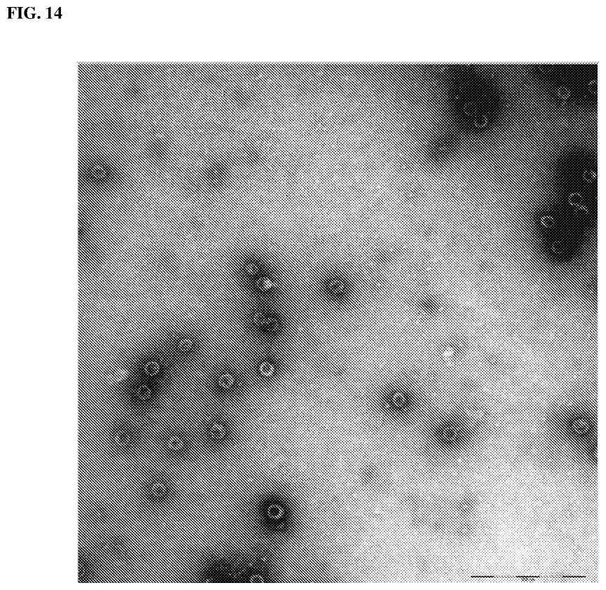

QBeta NPs conjugated to GBS saccharides were also characterized by transmission electron microscopy (TEM) analysis, using negative stain (NS). For analysis, NPs conjugated to GBS oligosaccharides and polysaccharides were loaded onto copper 300-square mesh grids of carbon/formvar (Agar Scientific) rendered hydrophilic by glow discharge (Quorum Q150). The excess solution was blotted off using Whatman filter Paper No.1 and then the grids were negatively stained with NanoW. Micrographs were acquired using a Tecnai G2 Spirit Transmission Electron Microscope at 87000× magnification equipped with a CCD 2k×2k camera. Negative stain TEM images of QBeta nanoparticles conjugated to GBS PSIa showed typical icosahedral symmetry with a diameter around 33 nm (FIG. 14).

In Vivo Immunization

A mouse immunization study was conducted, using GBS serotype Ia polysaccharide, either conjugated to CRM₁₉₇ carrier protein or to QBeta. The study included five groups of ten female mice each (CD1 strain, Charles River). Mice were immunized intramuscularly twice with the PSIa-CRM conjugates or once with PSIa-QBeta conjugates, as shown in Table 17. Immunizations were carried out on days 1 and 22 for PSIa-CRM and only on day 1 for PSIa-QBeta. Blood was drawn on days 0 (pre-immunization), 21 and 42.

TABLE 17

| | | | Dose-1st | Dose-2nd |
|---|---|---|---|---|
| Group | Sample | Adj | immunization | immunization |
| 1 | PSIa-CRM | Alum 2 mg/mL | 0.1 μg | 0.1 μg |
| 2 | PSIa-CRM | | 0.5 μg | 0.5 μg |
| 3 | PSIa-CRM | | 0.5 μg | 0.5 μg |
| 4 | PSIa-CRM | | 2 μg | 2 μg |
| 5 | PSIa-CRM | | 2 μg | 2 μg |
| 6 | PSIa-QBeta | | 0.1 μg | none |
| 7 | PSIa-QBeta | | 0.5 μg | none |
| 8 | PSIa-QBeta | | 0.5 μg | none |
| 9 | PSIa-QBeta | | 2 μg | none |
| 10 | PSIa-QBeta | | 2 μg | none |

Table 18 shows the Geometric Mean IgG Titers from individual sera belonging to each group of mice, along with Opsonophagocytic Killing Titers in pooled sera from each group of mice.

TABLE 18

| | | | | | Luminex IgG GMT Titer | | | | | |
|---|---|---|---|---|---|---|---|---|---|---|
| | | | | | in sera [RLU/mL] | | | OPKA titers | | |
| | Mice | | | | Day 21 | Day 42 | | Day 21 | Day 42 | |
| Group | CD1 | Sample | Adjuvant | Dose | post-1 | post-1 | post-2 | post-1 | post-1 | post-2 |
| 1 | 1-10 | PSIa- | Alum | 0.1 ug | 85 | na | 1278 | <30 | na | 424 |
| 2 | 11-20 | CRM | 2 mg/mL | 0.5 ug | 37 | na | 2060 | <30 | na | 162 |
| 3 | 21-30 | | | | 53 | na | 1704 | <30 | na | 162 |
| 4 | 31-40 | | | 2 ug | 46 | na | 1103 | <30 | na | 75 |
| 5 | 41-50 | | | | 34 | na | 670 | <30 | na | 42 |
| 6 | 51-60 | PSIa- | | 0.1 ug | 239 | 1458 | na | <30 | 139 | na |
| 7 | 61-70 | QBeta | | 0.5 ug | 280 | 2334 | na | 52 | 370 | na |
| 8 | 71-80 | | | | 324 | 2468 | na | <30 | 216 | na |
| 9 | 81-90 | | | 2 ug | 316 | 4501 | na | 87 | 292 | na |
| 10 | 91-100 | | | | 242 | 4293 | na | <30 | 465 | na |

Serum antibody titers were measured by a Luminex assay using streptavidin-derivatized magnetic microspheres (Radix Biosolutions, USA) coupled with biotinylated type Ia native polysaccharide (Buffi et al., (2019)). Following equilibration at RT, 1.25 million microspheres were transferred to LoBind tubes (Eppendorf) and placed into a magnetic separator for 2 min in the dark. Microspheres were washed with PBS containing 0.05% TWEEN™ 20 (Calbiochem) and biotin-PSIa was added to the microspheres at a final concentration of 1 μg/ml in PBS, 0.05% TWEEN™ 20, 0.5% BSA (Sigma-Aldrich). The biotin-PSIa—microspheres were incubated for 60 minutes at Room Temperature (RT) in the dark and washed twice with PBS, 0.05% TWEEN™ 20. Coupled microspheres were suspended in 500 μl of PBS, 0.05% TWEEN™ 20, 0.5% BSA and stored at 4° C.

Eight 3-fold serial dilutions of a standard hyperimmune serum or test samples were prepared in PBS, pH 7.2, 0.05% TWEEN™ 20, 0.5% BSA. Each serum dilution (50 μl) was mixed with an equal volume of conjugated microspheres (3,000 microspheres/region/well) in a 96-well Greiner plate (Millipore Corporation) and incubated for 60 min at RT in the dark. After incubation, the microspheres were washed three times with 200 μl PBS. Each well was loaded with 50 μl of 2.5 μg/ml anti-mouse IgG secondary antibody (Jackson Immunoresearch), in PBS, pH 7.2, 0.05% TWEEN™ 20, 0.5% BSA and incubated for 60 min with continuous shaking. After washing, microspheres were suspended in 100 μl PBS and shaken before the analysis with a Luminex 200 instrument. Data were acquired in real time by Bioplex Manager™ Software (BioRad).

The functional activity of the sera was determined by Opsonophagocytic Killing Assay (OPKA) as previously described (Chatzikleanthous (2020)). HL60 cells were grown in RPMI 1640 with 10% fetal calf serum, incubated at 37° C., 5% CO2. HL-60 cells were differentiated to neutrophils with 0.78% dimethylformamide (DMF) and after 4-5 days were used as source of phagocytes. The assay was conducted in 96-well microtiter plate, in a total volume of 125 μL/well. Each reaction contained heat inactivated test serum (12.5 μL), GBS Ia strain 515 (6×104 colony forming units [CFU]), differentiated HL-60 cells (2×106 cells) and 10% baby rabbit complement (Cederlane) in Hank's balanced salt solution red (Gibco). For each serum sample, six serial dilutions were tested. Negative controls lacked effector cells, or contained either negative sera or heat inactivated complement. After reaction assembly, plates were incubated at 37° C. for 1 hour under shaking. Before (T₀) and after (T₆₀) incubation, the mixtures were diluted in sterile water and plated in Trypticase Soy Agar plates with 5% sheep blood (Becton Dickinson). Each plate was then incubated overnight at 37° C. with 5% of CO₂; CFUs were counted the next day. OPKA titer was expressed as the reciprocal serum dilution leading to 50% killing of bacteria and the % of killing is calculated as follows

% killing=(T₀−T₆₀)/T₀

where T₀ is the mean of the CFU counted at T₀ and T₆₀ is the average of the CFU counted at T₆₀ for the two replicates of each serum dilution.

IgG and OPKA titers measured at day 42, after a single dose of PSIa-QBeta (groups 6-10), were non-inferior (comparable) to two doses of PSIa-CRM (groups 1-5).

### Example 11

### Conjugation of S. pneumonia Capsular Polysaccharide to NPs

Streptococcus pneumonia polysaccharide serotype 12F (Pn PS12F) was oxidized using (2,2,6,6-Tetramethylpiperidin-1-yl)oxyl (TEMPO) and trichloroisocyanuric (TCC). The oxidation was performed using 0.025 equivalents of TEMPO and 0.3 equivalents of TCC in NaHCO₃ 0.25M and Na₂CO₃ 0.025M buffer at pH 855. The oxidized polysaccharide was purified using a desalting column. Quantification and oxidation percentage of the resulting polysaccharide were assessed by HPAEC-PAD analysis.

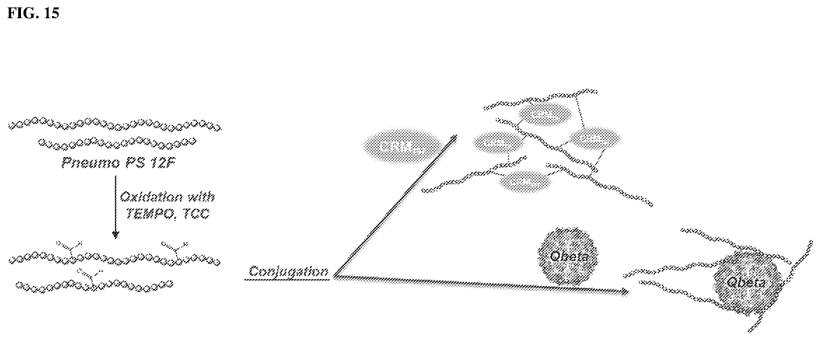

The oxidized polysaccharide was then conjugated to QBeta NP (5 mg/mL) at 37° C. for 72 hours by reductive amination in presence of NaBH₃CN, using a w/w ratio of saccharide to QBeta of 0.5:1. The final QBeta-Pn PS12F conjugate was purified by ammonium sulfate precipitation followed by serial centrifugal filtration (100 kDa). In parallel, the oxidized polysaccharide was conjugated to the monomeric carrier protein CRM197 using the same condition but a w/w ratio of saccharide to CRM197 of 1:1. The final CRM197-Pn PS12F conjugate was purified from free protein and saccharide by size exclusion chromatography (S500HP resin) (FIG. 15).

Pneumo PS12F-QBeta and -CRM197 Conjugates Characterization

HPAEC-PAD and BCA were used to estimate saccharide (total and free) and protein content, respectively, of purified Pn PS12F-QBeta and -CRM conjugates, as reported in the Table 19 below.

TABLE 19

| | | Sac- | | Saccharide/ | Free |
|---|---|---|---|---|---|
| NP | | charide | Protein | protein | saccharide |
| conjugate | Lot | (μg/mL) | (μg/mL) | (w/w) | % |
| PnPS12F- | FC26Feb21 | 215.3 | 1300.0 | 0.17 | 15.0 |
| QBeta | | | | | |
| PnPS12F- | FC05mar21 | 127.3 | 637.0 | 0.20 | <4.1 |
| CRM | | | | | |

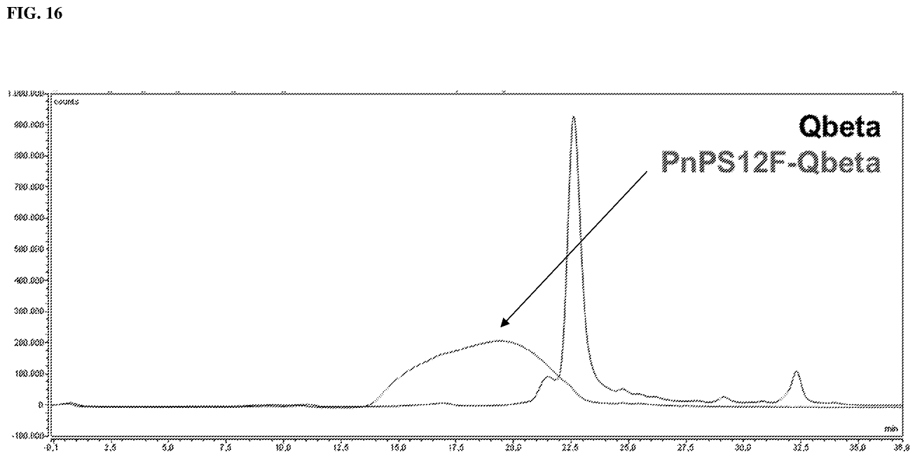

FIG. 16 shows the SE-HPLC analysis of Pneumo PS12F-QBeta NP conjugate and the QBeta NP (no conjugated saccharide).

SE-HPLC was carried out using SRT-C 2000 column with fluorometric detection (excitation at 227 nm and emission at 335 nm). Running conditions were flow rate 0.5 mg/mL, run time 40 minutes, 100 mM NaPi, 100 mM Na2SO4, pH 7.2 as running buffer and injection volume 20 μL. All samples were injected in a protein concentration of 0.3 mg/mL protein based.

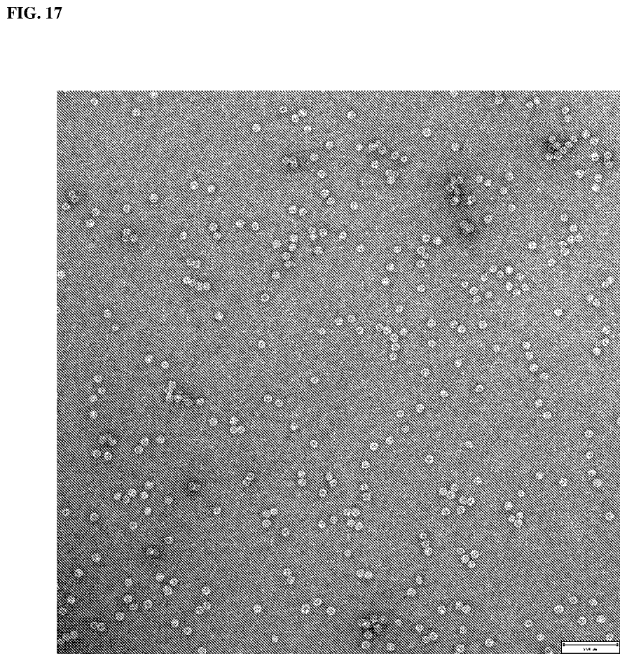

Pn PS12F-QBeta conjugate was also characterized by transmission electron microscopy (TEM) analysis, using negative stain (NS). For analysis, QBeta conjugate was loaded onto copper 300-square mesh grids of carbon/formvar (Agar Scientific) rendered hydrophilic by glow discharge (Quorum Q150). The excess solution was blotted off using Whatman filter Paper No.1 and then the grids were negatively stained with NanoW. Micrographs were acquired using a Tecnai G2 Spirit Transmission Electron Microscope at 87000× magnification equipped with a CCD 2k×2k camera (FIG. 17).

In Vivo Immunization

An in vivo mouse immunization study was conducted, using Pneumo serotype 12F polysaccharide, either conjugated to CRM₁₉₇ carrier protein, or conjugated to QBeta. Immunizations and blood draws were carried out according to the schedule in Table 20.

TABLE 20

| Day | Action |
|---|---|
| 0 | Blood Draw 1 (pre-immunization) |
| 1 | Immunization 1 |
| 21 | Blood Draw 2 (post first immunization) |
| 22 | Immunization 2 |
| 42 | Blood Draw Final (post second immunization) |

In the in vivo study, nine groups of ten female mice each (CD1 strain, Charles River) were studied. Each mouse was immunized intramuscularly once or twice with two different doses (0.1 and 1 μg, saccharide-based) of Pn PS12F-QBeta conjugate or Pn PS12F-CRM197 conjugates as shown in Table 21. Immunizations were carried out at Day 1 and Day 22. Blood was drawn from each mouse on Day 0 (pre-immunization), Day 21, and Day 42, as described in Table 20.

TABLE 21

| | | | Dose-1st | Dose-2nd |
|---|---|---|---|---|
| Group | Sample | Adj | immunization | immunization |
| 1 | PnPS12F-CRM- | Alum | 0.1 μg | 0.1 μg |
| | prep1 | 2 mg/mL | | |
| 2 | PnPS12F-CRM- | | 1 μg | 1 μg |
| | prep1 | | | |
| 3 | PnPS12F-CRM- | | 1 μg | none |
| | prep1 | | | |
| 7 | PnPS12F-QBeta | | 0.1 μg | 0.1 μg |
| 8 | PnPS12F-QBeta | | 1 μg | 1 μg |
| 9 | PnPS12F-QBeta | | 1 μg | none |

Serum antibody titers in serum were measured by a ELISA. Briefly, plates were coated with Pneumo polysaccharide serotype 12F and incubated with two-fold serial dilutions of sera followed by AP-conjugated secondary antibody. IgG titers were calculated by the reciprocal serum dilution giving Optical density (OD) equal to 0.5. Table 22 shows the Geometric Mean IgG Titers in sera as measured by ELISA for in vivo study.

TABLE 22

| | | | | | ELISA IgG GMT Titer in sera | | |
|---|---|---|---|---|---|---|---|
| | | | | | [RLU/mL] | | |
| | Mice | | | | post1_day2 | post1_day4 | post2_day4 |
| Group | CD1 | Sample | Adj | Dose | 1 | 2 | 2 |
| 1 | 1-10 | PnPS12F- | Alum | 0.1 ug | 5 | | 222 |
| | | CRM | 2 mg/mL | | | | |
| 2 | 11-20 | PnPS12F- | | 1 ug | 5 | | 68 |
| | | CRM | | | | | |
| 3 | 21-30 | PnPS12F- | | 1 ug | 4 | 21 | |
| | | CRM | | | | | |
| | | (one | | | | | |
| | | injection) | | | | | |
| 4 | 31-40 | PnPS12F- | | 0.1 ug | 22 | | 1269 |
| | | Qbeta | | | | | |
| 5 | 41-50 | PnPS12F- | | 1 ug | 30 | | 1436 |
| | | Qbeta | | | | | |
| 6 | 51-60 | PnPS12F- | | 1 ug | 20 | 1092 | |
| | | Qbeta | | | | | |
| | | (one | | | | | |
| | | injection) | | | | | |

After 21 days from the first dose, IgG titers in the PnPS12F-Qbeta conjugate groups were statistically superior (Mann-Whitney test) to those receiving PnPS12F-CRM conjugate. This difference became more evident comparing the responses obtained after 42 days, where the specific IgG anit-Pn12F elicited by one single shot of Qbeta conjugate was more than 10 fold to that obtained with 2 doses of the CRM conjugate.

### REFERENCES

WO 92/11291
WO 96/30523
WO96/40795
WO 98/15631
WO98/42721
WO02/056905
WO/03/024480
WO03/024481
WO2006/082530
WO2012/035519
WO2014/053612
WO2016/178123
WO2018/005558
WO 2018/229708
U.S. Pat. No. 4,356,170
U.S. Pat. No. 5,071,651
U.S. Pat. No. 5,306,492
U.S. Pat. No. 5,374,426
U.S. Pat. No. 9,657,065
U.S. Pat. No. 8,691,209
U.S. Pat. No. 9,950,055
European Patent No. 0477508
Ahmad & Chapnick, Infect Dis Clin North Am 13:113-33 (1999).
Altschul et al., J. Mol. Biol. 215:403 (1990).
Altschul et al., Nature Genet. 6:119, (1994).
Baker et al., Safety and immunogenicity of capsular polyaccharide-tetanus toxoid conjugate vaccines for group B streptococcal types Ia and Ib. J. Infect Dis. 179(1):142-150 (1999).
Baker et al., Use of capsular polysaccharide-tetanus toxoid conjugate vaccine for type II Group B Streptococcus in healthy women. J. Infect Dis 182(4) :1129-38 (2000).
Baker et al., Safety and immunogenicity of a bivalent group b streptococcal conjugate vaccine for serotypes II and III. J Infect Dis 188(1):66-73 (2003)
Baker et al., Immune response of healthy women to 2 different group b streptococcal type v capsular polysaccharide-protein conjugate vaccines. J Infect Dis. 189(6):1103-12, (2004).
Baker, Vaccine Suppl 4, D3-6 (2013)
Baker et al., Maternal antibody at delivery protects neonates from early onset group B streptococcal disease. J. Infect Dis. 209:781-8 (2014).
Bruun et al., ACS Nano 12(9):8855-8866 (2018).
Buffi et al., mSphere 2019 019 Vol 4 Issue 4 e00273-19 (2019).
Buttery & Moxon, J R Coll Physicians Lond 34:163-68 (2000).
Chatzikleanthous D, J Control Release. Apr 2;323:125-137 (2020).
Ciliens et al., FEBS Letters 24171:1-4 (2000).
Corpet et al., Nucleic Acids Research 16:10881 (1988).
Correira et al, Nature, 507, 201-6 (2014).
Cui et al., PNAS 114(44):11697-11702 (2017).
Current Protocols in Molecular Biology, John Wiley & Sons, N.Y. (1989), 6.3.1-6.3.6.
Dagan et al., Infect. Immun 66:2093-2098 (1998).
Dangor et al., Correlates of protection of serotype-specific capsular antibody and invasive Group B Streptococcus disease in South African infants. Vaccine; 33:6793-9 (2015).
Dick et al. in Conjugate Vaccines (eds. Cruse et al.) Karger, Basel, 10:48-114 (1989)
Donders et al., Maternal immunization with an investigational trivalent group b streptococcal vaccine: a randomized controlled trial. Obstetrics and Gynecology, 127(2):213-221 (2016).
Fabbrini et al., The protective value of maternal group B Streptococcus antibodies: quantitative and functional analysis of naturally acquired responses to capsular polysaccharides and pilus proteins in European maternal sera. Clin. Infect. Dis; 63:746-53 (2016).
Findlow and Borrow, Hum. Vaccin. Immunother, 12:226-230 (2016).
Freshney, Culture of Animal Cells, a Manual of Basic Technique, third edition, Wiley-Liss, New York (1994).
Goldblatt, J. Med. Microbiol. 47:563-7 (1998).
Golmohammadi et al., Structure 4:543-5554 (1996).
Heath and Schuchat, Perinatal group B streptococcal disease, Best Practice & Research Clinical Obstetrics and Gynaecology, 21(3):411-24 (2007).
Hermanson, Bioconjugae Techniques, Academic Press, San Diego, ISBN: 0123423368 (1996).
Heyderman et al., Group B streptococcus vaccination in pregnant women with or without HIV in Africa: a non-randomised phase 2, open-label, multicentric trial. Lancet Infectious Diseases, published on-line 8 Feb. 2016 (2016).
Higgins and Sharp, Gene 73:237 (1988).
Higgins and Sharp, CABIOS 5:151 (1989).
Hsia et al., Design of a Hyperstable 60-Subunit Protein Icosahedron. Nature, 535(7610):136-139 (2016); correction at Nature 540(7631):150 (Dec 2016).
Jardine et al., Science 340:711-716 (2013).
Jiang, et al., Science 250:1580-1583 (1990).
Kanekiyo et al., Nature 499(7456):102 (2013).
Kendrew et al. (eds.), The Encyclopedia of Molecular Biology, published by Blackwell Science Ltd., 1994 (ISBN 0-632-02182-9).
Kobayashi et al., WHO consultation on group B Streptococcus vaccine development: Report from a meeting held on 27-28 Apr. 2016. Vaccine (2016), available online 22 Dec. 2016 at http:// dx.doi.org/10.1016/j.
Kozlovska et al., GENE 137: 133-137 (1993).
Kwong et al. ACS Infect. Dis. 4(5):788-796 (2018).
Kyte et al., J. Mol. Bio. 157: 105-132 (1982).
LeRoux-Roels et al., A randomized, observer-blind Phase Ib study to identify formulations and vaccine schedules of a trivalent Group B Streptococcus vaccine for use in non-pregnant and pregnant women. Vaccine 34:1786-1791 (2016).
Lewin, Benjamin Genes V, published by Oxford University Press, 1994 (ISBN 0-19-854287-9).
Li et al., Ind. Biotechnol. 2:143-47 (2006).
Li et al., J Nanobiotechnol 17:13 (2019).
Lin, et al., Level of maternal antibody required to protect neonates against early-onset disease caused by group B Streptococcus type Ia: A multicenter, seroepidemiology study. J Infect Dis.184(8):1022-1028 (2001).
Lin et al., Level of maternal IgG anti-Group B Streptococcus type III antibody correlated with protection of neonates against early-onset disease cause by this pathogen. J Infect Dis. 190(5):928-934 (2004)
Lindberg, Vaccine 17 Suppl 2:S28-36 (1999).
Lopez-Sagaseta et al., Comput Struct Biotechnol J, 14:58-68 (2015).
Madhi et al., Safety and immunogenicity of an investigational maternal trivalent group B streptococcus vaccine in healthy women and their infants: a randomised phase 1b/2 trial. Lancet Infect Dis; 16 :923-34 (2016).
Madhi et al., Clin. Infect. Dis. 65(11):1897-1904 (2017).
Matsui, et al., J. Clin. Invest. 87:1456-1461 (1991).
Meyers, Robert A. (ed.), Molecular Biology and Biotechnology: a Comprehensive Desk Reference, published by VCH Publishers, Inc., 1995 (ISBN 1-56081-569-8)
Michon et al., Clin Vaccine Immunol. 2006 Aug;13(8):936-43 (2006).
Needleman and Wunsch, A General Method Applicable to the Search for Similarities in the Amino Acid Sequence of Two Proteins, J. Mol. Biol. 48:443-453 (1970).
New Trends and Developments in Vaccines, edited by Voller et al., University Park Press, Baltimore, Maryland, U.S.A. 1978
Nilo et al, ACS Chem Biol, 10, 1737-46 (2015a).
Nilo et al, Bioconj. Chem., 19, 1839-49 (2015b).
O'Dempsey et al. Meningococcal antibody titres in infants of women immunised with meningococcal polysaccharide vaccine during pregnancy. Arch Dis Child Fetal Neonatal Ed;74:F43-6 (1996).
Paoletti et al., J Biol Chem. 265:18278-83 (1990).
Paoletti et al., Infect Immun , 64(2):677 (1996).
Paoletti & Kasper, Expert Opin Biol Ther 3:975-84 (2003).
Pearson and Lipman, Proc. Natl. Acad. Sci. USA 85:2444 (1988).
Pharmaceutical Biotechnology, Vol.61, Vaccine Design-the subunit and adjuvant approach, edited by Powell and Newman, Plenum Press (1995).
Polonskaya et al, J Clin Invest. 127(4):1491-1504 (2017).
Quiambao et al., Immunogenicity and reactogenicity of 23-valent pneumococcal polysaccharide vaccine among pregnant Filipino women and placental transfer of antibodies. Vaccine; 25:4470-7 (2007).
Ramsay et al., Lancet 357(9251):195-196 (2001).
Remington's Pharmaceutical Sciences, by E. W. Martin, Mack Publishing Co., Easton, Pa., 15th Edition (1975).
Rice et al., EMBOSS: The European Molecular Biology Open Software Suite, Trends Genetics 16: 276-277 (2000).
Runnieks et al., Protein Sci 20:1707-1712 (2011)).
Sambrook et al., Molecular Cloning—A Laboratory Manual, 2nd Edition, Cold Spring Harbor Laboratory Press, 1989, pp. 9.31-9.57.
Shahid et al., Placental and breast transfer of antibodies after maternal immunization with polysaccharide meningococcal vaccine. Vaccine; 20:2404-9 (2002).
Smith and Waterman, Adv. Appl. Math. 2:482, (1981).
Stoll et al., J. Biol. Chem. 252:990-993 (1977)
Twomey, et al., Vaccine 13: 1603-1610, (1995).
Ulrich et al., Virus Res. 50:141-182 (1998).
Vasiljeva et al, FEBS Letters 431:7-11 (1998).
Voysey et al., Vaccine 34:3986-3992 (2016).
Wames, et al., Gene 160:173-178 (1995).
Wang et al., FEBS Open Bio 7(8):1196 (2017)).
Wessels et al., J Clin Invest 86:1428-33 (1990).
Wu et al., ACS Chem. Biol., 14:2176-2184 (2019).

1. A protein nanoparticle having an antigenic molecule conjugated to its exterior surface, wherein the antigenic molecule is a bacterial saccharide, and wherein the bacterial saccharide is a polysaccharide or an oligosaccharide.

2. The protein nanoparticle of claim 1, wherein the bacterial saccharide is selected from the group consisting of a Acinetobacter species, Bacillus species, Bordetella species, Borrelia species, Burkholderia species, Campylobacter species, Candida species, Chlamydia species, Clostridium species, Corynebacterium species, Enterococcus species, Escherichia species, Francisella species, Haemophilus species, Helicobacter species, Klebsiella species, Legionella species, Listeria species, Neisseria species, Proteus species, Pseudomonas species, Salmonella species, Shigella species, Staphylococcus species, Streptococcus species, Streptomyces species, Vibrio species, and Yersinia species.

3. The protein nanoparticle of claim 1, wherein the bacterial saccharide is from a Streptococcus species, wherein the Streptococcus species is Streptococcus agalactiae (“Group B Streptococcus” or “GBS”) or Streptococcus pneumoniae.

4. The protein nanoparticle of claim 1, wherein the bacterial saccharide is conjugated directly to the protein nanoparticle or via a spacer (linker) group.

5. The protein nanoparticle of claim 1, wherein the bacterial saccharide is conjugated to the protein nanoparticle by a method selected from the group consisting of (a) reductive amination; (b) carbodiimide chemistry (for example EDAC OR EDC); (c) maleimide chemistry; and (d) cyanylation chemistry (for example CDAP).

6. The protein nanoparticle of claim 1, wherein the protein nanoparticle is a non-viral protein nanoparticle.

7. The protein nanoparticle of claim 1, wherein the protein nanoparticle is a bacteriophage VLP, wherein the bacteriophage VLP is a Qbeta VLP.

8. The protein nanoparticle of claim 1, wherein the protein nanoparticle comprises a subunit polypeptide having at least 95%, at least 96%, at least 97%, at least 98%, at least 99%, or 100% sequence identity to any one of SEQ ID NO: 1, SEQ ID NO: 2, SEQ ID NO:3, SEQ ID NO: 4, SEQ ID NO: 5, SEQ ID NO: 6, SEQ ID NO: 7, or SEQ ID NO: 11, wherein the subunit protein is capable of self-assembling to form the nanoparticle.

9. The protein nanoparticle of claim 1, wherein the protein nanoparticle is capable of eliciting a higher immune response to the bacterial saccharide after one dose compared to after one dose of a monomeric protein carrier, such as CRM197, conjugated to the same bacterial saccharide.

10. The protein nanoparticle of claim 1, wherein the protein nanoparticle is capable of eliciting a higher or comparable immune response to the bacterial saccharide after one dose compared to after two doses of a monomeric protein carrier, such as CRM197, conjugated to the same bacterial saccharide.

11. An immunogenic composition comprising at least one protein nanoparticle according to claim 1.

12. The immunogenic composition of claim 11, further comprising an adjuvant.

13. A method of producing the protein nanoparticle of claim 1, comprising one or more of the steps of (a) culturing a recombinant host cell expressing the NP subunit polypeptide(s) of the invention under conditions conducive to the expression of the polypeptide(s) and self-assembly of the NP; (b) recovering or purifying assembled NPs from the host cell or the culture medium in which the host cell is grown, as is suitable; (c) extracting and purifying native polysaccharide from bacteria; (d) preparing bacterial oligosaccharides; and (e) conjugating bacterial polysaccharide or oligosaccharide antigen to the exterior of the NP.

14-15. (canceled)

16. The protein nanoparticle of claim 1, wherein the protein nanoparticle induces an immune response in a subject.

17. A method of inducing an immune response in a human subject, comprising administering to the subject an immunologically effective amount of the protein nanoparticle of claim 1.

18. A method of preventing or treating a bacterial infection in a human subject, comprising administering to the subject an immunologically effective amount of the protein nanoparticle of claim 1.

19. The method of claim 17, wherein the subject receives a single administration of the protein nanoparticle.

20. (canceled)

21. A method of inducing an immune response in a human subject, comprising administering to the subject an immunologically effective amount of the immunogenic composition of claim 11.
